(19) United States
(12) Patent Application Publication
(10) Pub. No.: US 2002/0197631 A1
(43) Pub. Date: Dec. 26, 2002

(54) Multichamber device and uses thereof for processing of biological samples

(76) Inventors: Nathan P. Lawrence, Timonium, MD (US); Feng Tao, German Town, MD (US); Allan Kakita, Huntington Beach, CA (US); Mark M. Manak, Laurel, MD (US); Richard T. Schumacher, South Easton, MA (US); James A. Laugharn JR., Winchester, MA (US)

Correspondence Address: JOHN W. FREEMAN, ESQ., Fish & Richardson P.C., 225 Franklin Street, Boston, MA, 02110-2804, US

(21) Appl. No.: 10/134,054

(22) Filed: Apr. 26, 2002

Related U.S. Application Data
(60) Provisional application No. 60/337,336, filed on Nov. 8, 2001.
(60) Provisional application No. 60/308,869, filed on Jul. 30, 2001.
(60) Provisional application No. 60/286,509, filed on Apr. 26, 2001.

(51) Int. Cl. C12Q 1/68; B01L 3/00; C12M 1/00

(52) U.S. Cl. 435/6; 422/99; 422/101; 436/177; 435/287.2

(57) ABSTRACT

Devices and methods are described for homogenization, processing, detection, and analysis of biological samples such as insects, fungi, bacteria, and plant and animal tissues. Multiple chambers in these devices permit different processing functions to be carried out at each stage, such that the resulting homogenized product can be further processed, purified, analyzed, and/or biomolecules such as metabolites, proteins and nucleic acids, or pharmaceutical products can be detected. The device can be used in a hydrostatic pressure apparatus, in which different activities, i.e. incubations, addition or renewal of reagent, and generation and detection of signal can be carried out in the appropriate chamber. The method improves the preservation of biomolecules from chemical and enzymatic degradation relative to conventional means. Additionally, this method enables automated sample preparation and analytical processes.

### FIELD OF THE INVENTION

The invention is in the general field of methods and multichamber devices for preparation (for example, homogenizing) and processing of biological samples, optionally in connection with analysis and/or detection of materials from a sample. Particular embodiments have applications in biotechnology, medical diagnostics, agriculture, food, forensic science, pharmaceutical, environmental and veterinary science.

### BACKGROUND OF THE INVENTION

Biological samples are frequently subjected to processing and analysis after they are isolated. Processing of such samples typically involves one or more of the following: homogenization of biological tissues, lysis of cells, suspension or dissolution of solid particulates, and liquefaction of solid material. Often, sample preparation also entails extensive enzymatic digestion, the use of harsh chemical reagents, and/or mechanical disruption. Following this initial preparation, the sample can be further processed using techniques such as polymerase chain reaction (PCR) or gel electrophoresis to purify or amplify particular molecules of interest such as nucleic acids and/or proteins in a sample. After processing, samples are typically subjected to an analytical and/or detection procedure.

Particular difficulties can be encountered in the application of molecular techniques to plant and animal tissues and to bacteria with rigid cell walls such as certain mycobacteria. Current methods for extracting biological molecules from such samples are limited by the requirement for complex processing using multiple steps and can be very time-consuming, labor intensive, and costly. Processing of bacteria or tissue, for example, can require extensive pretreatment with enzymes such as lysozyme or proteinase K, or grinding with glass beads. For some cells and tissues, additional mechanical disruption is also often necessary, requiring equipment such as a mortar and pestle, bead mills, a rotor-stator homogenizer, a blade blender, an ultrasonicator, a pulverizer, a pestle and tube grinder, a meat mincer, a Polytron®, or a French Press™. Extensive processing steps are required, for example, for the preparation and extraction of insoluble (inclusion-body) proteins, such as those produced by high-level expression of recombinant bacterial constructs.

Analysis of the biological properties of a sample can require further processing such as detection of nucleic acids, proteins, antibodies, factors, or activities extracted from the sample. Such further processing can require additional steps such as hybridization of nucleic acids with specific primers or probes, amplification, and detection of specific signals. For analysis of protein or antibody activity, binding to, or elution from specific ligands, antigen-antibody reactivity, or another “processing” system is sometimes required for identification and/or purification of the desired products. Examination of biological activity can also include incubation of an extract with a cascade of enzymes and/or co-factors to generate a detectable product. Gentle, yet effective procedures to release these molecules are desirable.

It is highly desirable to prepare, process, and analyze biological samples using equipment and procedures designed to simplify the overall process, to standardize the methods over a wide range of specimen types, to be amenable to automation, and to limit degradation of sample components, particularly biomolecules.

Simplification or automation of sample preparation steps can save time and money, and can result in a more reliable output from analytical techniques, as a result of, for example, reduced manual handling of the sample.

### SUMMARY OF THE INVENTION

The invention described allows for various purification and/or analytical steps to be carried out in the chambers of a multi-chamber device, which is amenable to automation, where at least one of the desired processes is achieved using high pressure.

The invention is based, at least in part, on the discovery that application of cycled pressure, variable temperatures, or both, to a sample in a device having multiple chambers separated by penetrable and/or porous barriers can allow biological samples to be processed in a controlled and automated manner. While the new device is amenable to use with liquid samples, it can also be used in a similar manner with solid and semi-solid samples, such as whole plant or animal tissue, and gaseous samples.

The new devices and methods provide a simple format for loading and unloading samples and the methods generally can be automated while still effectively fragmenting, homogenizing, and processing tissue samples. The methods and devices reduce the necessity to transfer samples and reagents as the samples are generally processed sequentially. In one embodiment of the invention, large chunks of tissue are first fragmented into smaller pieces and then homogenized to completion, while avoiding unacceptable degradation of targeted biomolecules, before the sample is further processed. Release of biomolecules such as nucleic acids or proteins is efficient, and the function of those molecules (e.g., in the lysate) is well preserved. After the sample has been homogenized and the biomolecules have been released, the sample undergoes further processing, typically after being forced into a subsequent chamber of the multichamber device. Further processing of the sample can include, but is not limited to, one or a combination of the following processes: purification by chromatography, solid phase capture, or gel electrophoresis; enzymatic processing; amplification of biomolecules (e.g., PCR); processing and/or detection of protein interactions; chemical modification; substrate labeling; solublization of substrate; and substrate detection.

The homogenization aspect of the method is readily used in conjunction with high hydrostatic pressure cycling technology (“PCT”). The use of PCT technology to analyze biological samples is generally described elsewhere, for example in the following documents which are incorporated by reference in their entirety: Laugharn, Jr. et al., U.S. Pat. No. 6,111,096; Laugharn, Jr. et al., U.S. Pat. No. 6,120,985; Hess, R. and Laugharn, Jr., U.S. Pat. No. 6,127,534; Hess, R. and Laugharn, Jr., U.S. Pat. No. 6,245,506; Laugharn, Jr. et al., U.S. Pat. No. 6,258,534; Laugharn, Jr. et al., U.S. Pat. No. 6,270,723; Laugharn, Jr. et al., U.S. Pat. No. 6,274,726 and PCT Application No. WO99/22868. Physical changes effected on the sample can include pressure-driven phase changes, volume changes under high pressures and homogenization, for example, as solids or other matter pass through screens. Such processes are readily adaptable to automation.

In one embodiment, the invention features a sample preparation device for use in a pressure modulation apparatus. The device includes multiple chambers, at least one barrier positioned between two chambers, and at least one pressurizing member such as a ram, positioned to subject the sample to high pressure and to force the sample through at least one barrier. The barrier can be either porous or penetrable (e.g., a septum) or can be rendered porous or penetrable by exposure to a physical, chemical, or mechanical stimulus. One or more of the chambers can be part of a single, one-piece, molded device.

In some cases, a barrier such as a screen or a shredder can be positioned between a first chamber and a second chamber, and openings in the barrier can allow communication of materials (e.g., solids, semi-solids, liquids, or gases) between the first chamber and the second chamber.

In other cases, the device can include three or more chambers with multiple barriers positioned between the chambers. At least one of the barriers can be porous or penetrable, or can be rendered porous or penetrable by exposure to a physical, chemical, or mechanical stimulus. The device can further include two or more pressurizing members that can be activated independently, e.g., at differing hydrostatic pressures. At least one pressurizing member, for example, a ram, is in a position to subject the sample to high pressure and to force the sample through at least one barrier. Two or more of the chambers can be linked together by threaded mechanical interlocks or a threading mechanism.

The chambers can be made of plastic, rubber, ceramic, metal, or glass or any combination of these materials. For example, the chamber can contain a volume up to about 100 μl, up to about 500 μl, up to about 1 ml, up to about 100 ml, or up to about 500 ml. The surface of one or more chambers can be rendered inert to biomolecules. The surface of one or more chambers can also be derivatized with biomolecules or small organic molecules such as pharmaceutically active compositions or metal chelators, through covalent bonding, ionic interactions, or nonspecific adsorption. In some cases, at least one barrier is pierceable (i.e., can be punctured with a sharp object), at least one barrier is capable of being dissolved by organic solvent (e.g., a poly(methyl methacrylate) barrier, which can be dissolved by, for example, tetrahydrofuran or ethyl acetate), at least one barrier can be removed mechanically (e.g., by the action of the ram or other device), at least one barrier can be removed through a change in temperature (e.g., a wax barrier can be melted by heating; a porous ceramic barrier clogged with a low-melting polymer can be rendered porous by heating), at least one barrier can include a valve (e.g., a one-way valve), or at least one barrier can be removed by any one or a combination of the following mechanisms: piercing, salvation, melting, breaking, and mechanical removal (e.g., unscrewing or prying). The barrier(s) can be made of a polymer, metal, or ceramic. The barrier can also be made up of a composite or layers of solid materials (e.g., sand or silica gel). The barrier can also include a filter for either capturing or excluding desired substrates for purification, or analysis. The filter(s) can be made of polyvinyl chloride, polyether sulfone, nylon, nitrocellulose, cellulose esters, cellulose acetate, cellulose nitrate, polyfluorethylene (PFTA), vinyl, polypropylene, polycarbonate or other material. The barrier(s) can, for example, have a plurality of openings, which can be, for example, round, and can have a diameter of between about 1 μm and about 3 cm (e.g., between about 1 μm and about 100 μm, between about 0.1 mm and 1 mm, between about 1 mm and 1 cm, between about 1 cm and 3 cm, or any combination of such ranges or intermediate range). The barrier can also include a plurality of solid, pointed protrusions, made, for example, of a polymer, metal, or ceramic. The pointed protrusions can be, for example, in the shape of a pyramid or cone, and can, for example, extend about 0.01 to 3 cm (e.g., about 0.01 cm, 0.1 cm, 0.2 cm, 0.5 cm, 0.8 cm, 1 cm, 1.5 cm, 2 cm, 2.5 cm, or 3 cm, or any intermediate range) above the base of the screen.

The pressurizing member can include (or can be), for example, at least one ram mounted to move relative to the chamber, and can be, for example, made of a polymer, metal, or ceramic. The pressurizing member can be, for example, circular in cross-section (e.g., the member can be cylindrical or conical). The chambers can, for example, include a wall, and the pressurizing member can include one or more annular seals (e.g., rubber or teflon O-ring) that contact the wall as the pressurizing member moves. The seal can be, for example, made from a polymeric, metallic, or ceramic material. The pressurizing member(s) can, for example, be a cylinder having a sealing ring around its circumference, in which case the chambers can be generally cylindrical. In some cases, the device can include at least two pressurizing members, one of the pressurizing members being positioned on a first side of a barrier and the other of the pressurizing members being positioned on a second side of the barrier, opposite to the first side of the barrier. More than one chamber can be mounted with a pressurizing member. (See FIG. 15.)

The device can also include a container having an orifice, in which case the chambers can be positioned within the orifice. One or more of the chambers can be filled with a fluid. One or more of the chambers can also include a temperature-controlling device, a temperature-cycling device, a pressure-controlling device, a pressure-cycling device, or an optical sensor.

The multiple chambers of the devices can also, optionally, be interconnected within the pressure-modulation apparatus. In some cases, a number of devices (e.g., 2, 3, 4, 6, 8, 10, 12 or more devices) can be interconnected to form a strip, or can be interconnected to form a two-dimensional matrix (e.g., an 8×12, 4×4, 2×2, 4×6, or 3×5 array).

In some cases, one or more chambers containing a reagent can annularly surround a chamber containing a sample, in which case the reagent can be introduced to the sample through a valve activated by pressure.

At least one of the chambers can also be equipped with electrodes to enable an electric current to be passed through the chamber during processing of the sample.

In another embodiment, the invention features a sample-processing device for use in a pressure-modulation apparatus. The device includes multiple chambers, positioned in a vertical configuration; multiple temporary barriers positioned between pairs of chambers; and a pressurizing member positioned to force a sample through the barriers. (See FIG. 14) Optionally, the device can also include a porous barrier positioned between the first and second chamber.

In still another embodiment, the invention features a method of processing a biological sample. The method includes providing a device as described above; adding the sample to a first chamber of the device; and subjecting the device to an elevated pressure (e.g., at least 100 psi, 250 psi, 500 psi, 750 psi, 1,000 psi, 5,000 psi, 10,000 psi, 20,000 psi, 30,000 psi, 40,000 psi, 50,000 psi, 60,000 psi, 70,000 psi, 80,000 psi, 90,000 psi, 100,000 psi, or more) to cause the pressurizing member to force a sample through a barrier between the first chamber and second chamber and into the second chamber.

The sample can be, for example, forced through multiple barriers by the pressurizing member. The biological sample can include, for example, a solid material, a semi-solid material, and/or a liquid. In particular embodiments, the biological sample can include, for example, an insect or small animal, a fungus, plant or animal tissue, a food product or agricultural product, a forensic sample, a human tissue (such as muscle, tumor, or organ), serum, sputum, blood, or urine. The biological sample can, optionally, be frozen. The size of the biological sample can be, for example, between about 0.1 mg and 500 g (e.g., 0.1 mg to 1.0 mg, 1.0 mg to 10 mg, 10 mg to 100 mg, 100 mg to 1 g, 1 g to 20 g, 20 g to 100 g, 100 g to 200 g, 200 g to 500 g).

The elevated pressure can be, for example, applied to the sample at, above, or below ambient temperature, for example, at ambient temperature, at a temperature below which the sample would freeze at atmospheric pressure (i.e., the sample's atmospheric pressure freezing temperature), at a temperature in the range of that temperature to about 4° C., between about 4° C. and ambient temperature, at a temperature in the range between ambient and 90° C. or higher (e.g., −80° C., −40° C., −20° C., 0° C., 4° C., 10° C., 15° C., 20° C., 25° C., 30° C., 37° C., 40° C., 50° C., 60° C., 70° C., 80° C., 90° C., 100° C., or higher).

The elevated pressure can also be repeatedly cycled (e.g., a frequency in the range of milliseconds (i.e., 1-10000 Hz), at a frequency in the range of seconds (0.01-1 Hz), or at a frequency in the range of minutes (e.g., 0.1-10 mHz).

The method can also include the steps of analyzing the sample after, or as part of, sample preparation, extracting a specific substance or substances from the biological sample, and/or processing a specific substance or substances from the biological sample. For example, DNA, RNA, proteins, or pharmaceutical compositions (e.g., pharmaceutically active molecules, natural products, drugs, or drug metabolites) in the sample can be isolated after or as part of the sample preparation; and/or a portion of the sample (e.g., a nucleic acid) can be amplified (e.g., using the polymerase chain reaction, “PCR”, or ligase chain reaction, “LCR”), bound to a ligand, eluted from a ligand, sequenced, or hybridized; and/or a portion of the sample can be subjected to chemical reactions including but not limited to antigen-antibody interactions, enzymatic reactions, catalytic reactions, or step-wise reactions wherein a different step takes place in each chamber of the device.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a cross-section of a device for preparing biological samples.

FIG. 2 is an illustration in perspective of a multichamber device assembly that contains two rams.

FIGS. 3A-3B depict two examples of barriers having shredder surfaces. The top shredder surface (3A) is composed by simple circular holes. The bottom shredder surface (3B) has not only holes, but also hard sharp protrusions between the holes, which aid in sample disruption.

FIGS. 4-12 depicts various gel electrophoresis results described below.

FIG. 13 is an illustration of a sample preparation device with shredder component, which fits in one well of a 96 well plate.

FIG. 14 is a cross-section of a sample preparation device having one porous barrier, multiple penetrable barriers, and one pressure modulation apparatus.

FIGS. 15A-15F are illustrations in a simplified schematic diagram of a sample preparation device having multiple penetrable barriers and multiple pressurizing members, which are rams.

FIGS. 16A-16B are illustrations of a top cross sectional view and side cross sectional view of a device, wherein sample or reagents are introduced into sample compartments both vertically and horizontally.

FIGS. 17A-17B are illustrations of a device capable of extracting nucleic acids using pressure and electrical current.

FIGS. 18A-18B are illustrations of a device having sample delivered into various compartments that are divided into portions of a cylinder. FIG. 18A illustrates the relative positions of the sample chamber and wash chamber. FIG. 18B illustrates how the sample is delivered through valves and the lower compartments move mechanically in a circular motion.

FIG. 19 is a cross-section of a multichamber device of the invention having double O-rings.

FIG. 20 is a drawing of a tool for setting the cap and ram of the device of FIG. 24.

FIG. 21 is a photograph of an agarose electrophoresis gel, showing genomic DNA purified from rat tails processed with the devices of the invention.

FIGS. 22A-22C are photographs of agarose gels (FIGS. 22A and 22B) of DNA and a Western blot (FIG. 22C) of proteins extracted from rat brains processed with the devices of the invention and other methods.

Like reference symbols in the various drawings indicate like elements.

### DETAILED DESCRIPTION OF THE INVENTION

A pressure-driven cell lysis method is described in U.S. Pat. No. 6,111,096 and the entirety of that patent is incorporated herein by reference. This invention features a device having multiple compartments (i.e., chambers) separated by one or more permeable or penetrable barriers capable of being subjected to high pressure. The invention is used to prepare organic or inorganic samples by homogenizing the samples and, in some embodiments, subjecting the samples to additional processing or analysis. The new devices can be used, inter alia, for sample collection, transport, processing, and/or storage, and for growth of organisms.

In one embodiment of this patent application, the invention features a device for insertion into a pressure-modulation apparatus. The basic components of the device include multiple compartments/chambers, one or more barriers separating the compartments, a cap, and one or more pressurizing members (e.g., rams).

Various volumes of compartments can be implemented with capacity appropriate for both the processing occurring in the particular compartments and the sample size. Capacity typically ranges from 25 microliters to 1 liter or greater (e.g., 25 μl, 50 μl, 100 μl, 250 μl, 500 μl, 1 ml, 2 ml, 5 ml, 10 ml, 15 ml, 20 ml, 25 ml, 30 ml, 40 ml, 50 ml, 75 ml, 100 ml, 250 ml, 500 ml, 750 ml, or 1 l, or intermediate volumes). The compartments can be positioned within the device in either a horizontal or vertical manner or a combination of the two. Additionally, the compartments of the device can be formed into a single unit or can be connected together modularly, for example, positioned in a 96-well format.

In some embodiments, the invention features at least one porous barrier such as a screen or shredder for preparing, particularly homogenizing, biological samples such as tissues in semisolid or solid forms, and generating a finely minced tissue or slurry from the whole tissues. This homogenization aspect of the invention includes either or both of two basic processes, primary fragmentation and subsequent homogenization of the sample. If desired, the homogenized sample can be further processed directly within the device without need for removal. The initial homogenization/processing can be further combined with standard extraction or analytical procedures utilizing, for example, enzymes, detergents, denaturants, or chaotropic salts and the like.

The porous barrier used in the invention can be made from solid material such as polymer, metal, glass, or ceramic. The orifices can contain sharp edges to facilitate the fragmentation of a solid sample. The pressurizing member can serve as a piston with one or more seals that deliver sample from one side of the barrier to the other while acting as an effective shield between the sample inside the device and the pressurizing medium outside. This process is defined as the primary fragmentation. When pressure is cycled, the primary fragmentation process is repeated to generate homogeneous slurry.

The device can process various quantities of specimen, typically from 10 milligrams up to at least 15 grams for analytical applications, and can be scaled up even further for preparative work. Pressure applied in the primary fragmentation can be relatively low, on the order of 10,000 to 50,000 psi (pounds per square inch). One or more of the compartments can be preloaded with appropriate lysis, capture, or processing solutions in predefined volumes. The specifics of the porous barrier design are determined in part by the volume of the lysis/capture/reaction fluid utilized and nature of the specimen, e.g., blood, urine, serum, liver, brain, skeletal muscle, plant leave, stem, root or animal tooth or bone. An appropriate device for the particular specimen is used (i.e., appropriate screen or porous barrier, container, lysis/capture/reagent fluid) and pressure cycling conditions are selected for sample characteristics of the sample size being processed and for the downstream analysis to be performed for that sample.

The device can contain additional compartments separated by non-porous, penetrable barriers. The compartments can, for example, contain reagents suitable for purification, reaction, or analysis of the sample. The surface of one or more compartments can be coated with material, making the surface inert to biomolecules; or the surface can be derivatized (i.e. covalently attached or ionically bonded) with biomolecules or molecules capable of interacting with biomolecules. The sample can be forced into the additional compartments in a sequential manner using pressure from the pressure modulation apparatus. For example, pressure can be continually increased, forcing the sample into consecutive adjacent compartments. Cycling of the pressure causes the sample and reagents in the subsequent chambers to become sufficiently mixed together. Each consecutive compartment can expose the sample to an additional step of processing. The number of consecutive compartments required in each device varies depending on the degree of processing desired for each individual sample.

While pressure is used to force the sample through the device, the barriers can also be penetrated under a variety of other conditions. For example, the barriers can be temperature-sensitive (e.g., wax barriers, or porous ceramic barriers, wherein the pores are initially clogged with wax), breaking down at a set temperature. The barriers can also be penetrated by exposure to solvents, causing the barrier to dissolve into solution. Additionally, the barriers can be valves, removed either mechanically or electronically.

After selecting the appropriate barriers and reagents, the sample can be loaded into the holder and one or more pressurizing members (e.g., rams) installed as required by the format of the device. The device is then placed inside a pressure cycling apparatus that is pre-equilibrated to the appropriate temperature. The selection of the barriers and the appropriate pressure cycling profile can be customized to the specimen requirements, e.g., based on the principles illustrated below.

One manner of processing amenable to the devices and methods of the present invention includes mechanical fragmentation of the tissue sample. Pressure is localized around the sharp edges or points incorporated into the screen. The sample is driven across the screen (usually in multiple passes) by the application of pressure pulses. The pressure difference between the sample and the capture fluid in the adjacent compartment is small (e.g., in the range of less than several hundred pound per square inch, resulting in a much gentler treatment of the tissue and better retention of biological structure and activity. Because the pressure drop across the porous barrier is relatively small, large molecules such as, for example, DNA and RNA, are not subjected to harsh mechanical shearing forces. The process is suitable for automation and can be performed at reduced temperature (well below standard cold room temperatures, which typically are about 4° C.) to improve retention of intact molecules, such as nucleic acid molecules.

Most liquids are partially compressible under high pressure and expand when pressure is released. The sample can initially be repeatedly pushed through a barrier (e.g., a shredder or a screen) during a first pressure cycling process. With each cycle, upon releasing pressure, unmacerated sample can be pushed back to the sample-loading compartment. During the process, lysis/capture solution can penetrate the sample and help liberate and solubilize the biomolecules inside the tissue sample.

Another form of processing, homogenization, can also be achieved by applying cyclic pressure, resulting in perturbations of large, multi-molecular structures (such as cell membranes) leading to their destruction. The homogenization process can also be applied to material that has already been fragmented by the methods described above or by other methods, or to frozen material, which can become a slurry by introduction of repeated phase changes such as liquid/solid (frozen) phase changes, further contributing to cell breakage. The low temperatures can help preserve biological activity during the process. This process is particularly practical and effective, for example, when applied to small fragments of semi-solid and solid samples.

The PCT process described here contributes to cell disruption by a variety of mechanisms in addition to those described above. Successful practice of the invention does not depend on any particular theory of its operation.

The homogenization can also be achieved by carrying out pressure cycling at relatively high temperatures, such as 50° C. to 90° C. or at moderate temperatures such as −5° C., 0° C., 4° C., 10° C., 20° C., 25° C., 30° C., 37° C., or 45° C. The mechanism of high temperature homogenization can be different from that at low temperature, as the solubility of biomolecules such as lipids and polysaccharides can be increased at the high temperature. There is a potential problem in that the degradation of biomolecules can increase at the higher temperatures and can be further exacerbated by increased activity of proteases or nucleases at the high temperatures. Nevertheless, a high pressure process at high temperature can be suitable and even preferable for certain stable proteins, nucleic acids, and other molecules at appropriate temperatures and pressures. For example, RNases can be inactivated to generate high yield and high quality RNA without the use of harsh chemicals.

The PCT method uses hydrostatic pressure or mechanical pressure applied uniformly to the sample in a closed container. The PCT device can accommodate an intact tissue sample. The multiple holes in the barrier (e.g., shredder) and wider diameter holes do not result in high shear or sudden pressure drops across the openings, but rather a more gentle and controlled passage of materials with minimum shearing and denaturation.

Another embodiment of the invention includes, having air trapped inside the chamber that occurs with the loading of the sample into the device. When pressure from the pressuring member increases, the air is compressed and can be dissolved into the samples. With a rapid release of pressure, the air can expand its volume rapidly, which can introduce disruptive effect on samples.

This rapid, yet gentle process allows efficient release of cellular contents while maintaining the integrity and biological activity of the liberated molecules in part because this process does not require the use of detergents, harsh chemicals, or excessive shearing forces. Alternately, the device can be used with detergents and other chemicals, which are compatible with maintenance of biological activity or where maintenance of biological activity is not necessary or not desired. Additionally, this process is well suited for the study of protein content and activities within cells and tissues. Important features of the current method that contribute to protein stability include low shear, rapid lysis, and high protein concentrations. Additional stabilizing additives such as proteinase inhibitors, glycerol, DTT, or specific cofactors can be added to the buffer to further protect the integrity of the desired proteins. The biological activities of many enzymes, particularly of monomeric proteins, remain fully functional after treatment. These proteins are suitable for subsequent purification and analysis by 1-D and 2-D gel electrophoresis, mass spectroscopy, and enzymatic activity assays. The ability to rapidly and efficiently isolate biologically active proteins from a variety of cells and tissues can have very wide applications in research, and in the medical, pharmaceutical, and biotechnology industries.

Important applications of the device and method include, but are not limited to:

1. Liberation and solubilization of recombinant proteins from inclusion bodies produced in microbes (e.g., E. coli)or from plant recombinant systems.

2. Isolation of intact proteins from biopsy specimens of tumor tissues (e.g., plant or animal tumor tissues), providing a significant advance in mapping and identification of tissue specific marker of early cancer events. Such markers can be used for diagnostics and early identification of specific cancer types.

3. Release of prions from brain or other tissues.

4. Identification of biomarkers that have the potential to effectively monitor drug efficacy and safety, based on data derived from the study of biological tissues.

5. Rapid extraction of proteins from a variety of medicinal plants, fungi, and other organisms, and facilitation of screening of organisms for sources of potential new drugs against cancers, infectious, and genetic disease, and other therapies.

6. Facilitation of adequate quantities of proteins for toxicological and animal tests in early stages of drug discovery.

7. Extraction of lead or other heavy metals, drugs, pesticides, or other inorganic or organic chemicals or components from sources described elsewhere in this application (e.g., organic materials such as plant or animal tissue or inorganic materials such as soil). Such extraction can be useful, for example, for environmental monitoring, tracking releases of hazardous materials (e.g., due to chemical spills or bio-terrorism), or uptake by an organism.

In certain embodiments, the device can include additional compartments or chambers that further process the homogenized sample. The compartments can be positioned adjacent to one another and can contain a variety of ingredients suitable for sequential processing of the sample as the sample is forced through the consecutive chambers within the device. For example, a compartment can contain capture elements used to purify the sample such as various binding ligands or solid phase particles. A compartment can also contain a variety of chemicals. These chemicals can be enzymes, substrates required for polymerase chain reaction (PCR), acids or bases, catalysts, and/or other biomolecules capable of processing the sample. Additionally, the compartments can be charged with a label used for analysis of the sample.

The surface of the compartments can be derivatized in a manner appropriate for the sample and the desired processing. In one embodiment, the surface can be coated with a material causing the surface to be inert to biomolecules, preventing attachment or adsorption thereof. In another embodiment, the surface can be covalently bonded or ionically attached to biomolecules or pharmaceutical products (i.e., small organic compounds) capable of interacting with or trapping elements of the homogenized sample.

In another embodiment, one or more of the compartments is equipped with a device (e.g., a photometer) that enables analysis and/or detection of the sample. Forms of analysis include but are not limited to measurement of temperature, pressure, radiation, absorbance, fluorescence, or turbidity.

The materials processed by this device cover a broad spectrum of specimen types. Examples are provided below. The sample types that can be processed include, but are not limited to, blood, serum, forensic samples, fungi, insects, plant tissue (e.g., pollen, leaves, roots, flowers or other plant parts, whether fresh, frozen, or dried), and animal tissue (e.g., avian, reptilian, fish, or mammalian tissue such as human, bovine, canine, feline, murine, or porcine tissue. Tissues can include biopsy specimens, crops or foods.

In summary, advantages of the present invention over the conventional methods include, inter alia:

Frozen samples can be processed without thawing.

One step fragmentation, homogenization, and processing can be carried out with minimum hands-on operation.

Lysis protocols can be tailored for specific biological samples, having the materials necessary for analysis and suitable for follow-up assays pre-loaded into the device.

Sample size can be flexible ranging from milligrams to hundreds of grams and the sample can be in whole pieces.

The process can be applied to a broad spectrum of sample types, such as liquid, solid, or semi-solid tissues.

The lysing and further sample processing and or analysis can be carried out in a handsoff, automated pressure cycling instrument.

The homogenization process can be rapid without the need for lengthy incubation such as that requires enzymatic digestion steps.

The process can include an automated molecular extraction procedure, such as, automated nucleic acid extraction method (see, e.g., U.S. Pat. No. 6,111,096), or more conventional extraction methods within a single device.

The method can be carried out at subzero temperatures (i.e. >0° C.) so that the integrity of biological molecules, such as RNA and enzymes, are preserved from degradation and remain functional.

Samples can be processed in a closed disposable holder, preventing cross-contamination of specimens. Specimens can be collected in the field, placed and stored in the device until processing, minimizing specimen handling.

The process can be adapted for simultaneous or sequential processing.

The device can be adapted to process multiple samples, for example, by being set-up in a matrix such as a 96-well format.

The process can be adapted for many applications, such as, forensic, clinical, pathological, agricultural, food safety, pharmaceutical, bio-terrorism and environmental analysis.

Organisms can be grown, processed, analyzed, and/or rendered inert within the device (e.g., a device containing a growth or transport medium). Of special importance when working with potentially dangerous or fastidious organisms, these methods can be carried out either with or without the need to open the device between sample collection/loading and rendering inert, thus minimizing possible hazards associated with handling of such organisms.

Whole, intact, and viable microorganisms and extracts thereof can be extracted from plant or animal sources or from inorganic substances such as soil.

Part 1. The Two-Chamber Homogenization Module

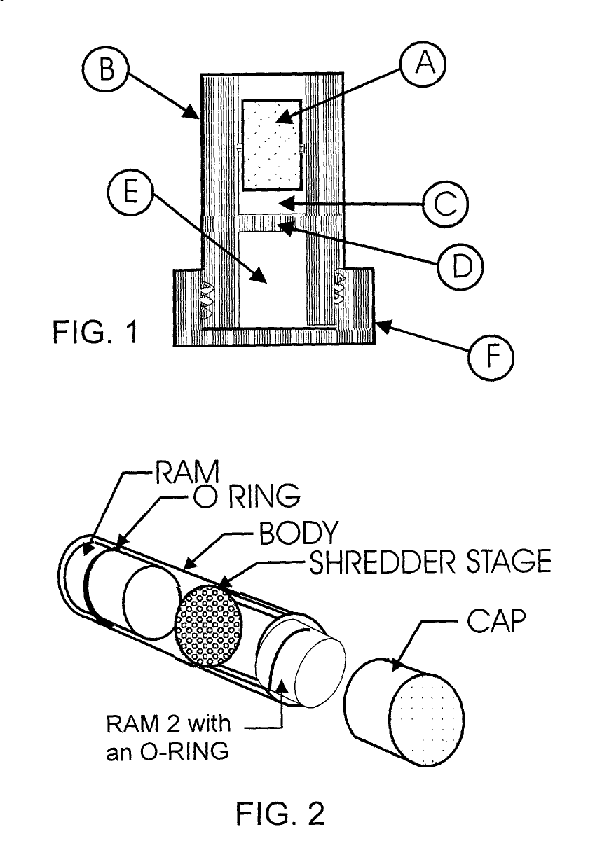

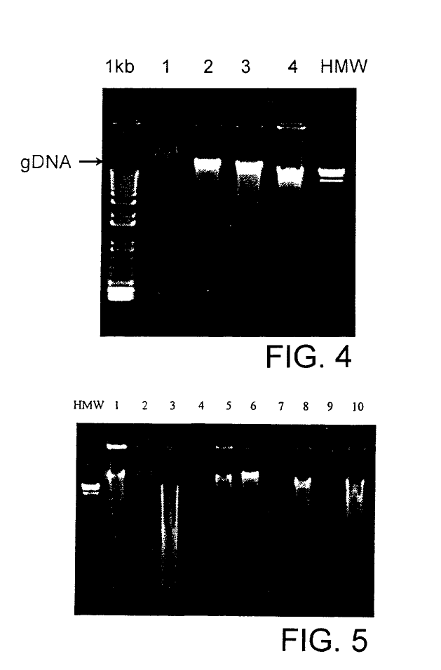

One design for the sample-processing device is a two-chamber module as illustrated in FIG. 1. This design has as its components a pressurizing member, which is a ram (A), a compartment having two chambers (B), barrier, which can be either a shredder screen or porous barrier (D), and a cap (F). In another design, the device can also have a second pressurizing member (e.g., a ram) (FIG. 2) with an O-ring. This homogenization module is adapted to fit into a pressure-cycling apparatus such as those described in U.S. Pat. No. 6,111,096 at pages 4-9 and 29-44 and in FIGS. 1 and 4.

Prior to loading the sample, the optional second ram can be inserted into the module. With the module placed upright, a defined volume of capture fluid added and the barrier (e.g., a screen) can be inserted from the top. The pressure compartment of the pressure-cycling apparatus and module can optionally be pre-chilled to a defined temperature, such as −20° C. or −30° C. The sample can then be placed into the sample chamber. The upper pressurizing member is positioned into the module body over the sample. The module is then placed into the compartment of the pressure cycling instrument where it is immersed in the temperature-equilibrated pressurizing fluid. The pressure compartment is then sealed.

As the pressure is increased in the chamber, the pressurizing member in the module moves towards the interior of the module of the first chamber, forcing the sample through the orifices in the barrier (e.g., a shredder screen) to the second, sample-capture chamber, where it becomes mixed with the capture fluid. In that process, the air between the top pressurizing member and the capture fluid may become dissolved in the liquid. Once the tissue sample is pressed by the pressurizing member driving towards the screen, the liquid volume of the combined tissue and capture fluid will decrease, down to, for example, 85-90% of the volume at atmospheric pressure. As the pressure is reduced, the tissue/capture fluid mix plus trapped air expands, force the top pressurizing member back up, and moving the mixture back through the screen. As the pressure within the chamber is cycled, the combined action of the pressure cycling and movement of the pressurizing member can force the solution repeatedly through the screen, contributing further to the physical break up of the cells and tissues.

The pressurizing member can be designed to transfer maximum pressure to a sample that is placed in the module body. The pressurizing member can be made of a hard material such as metal (e.g., titanium, stainless steel, or aluminum), plastic (e.g. thermoplastic such as polypropylene, p-phenylene sulfide, or glass reinforced PEI resin), glass, stone, or a ceramic material. The surface of the pressurizing member or the barrier at the point of contact can be designed to further assist in the disruption of the sample, e.g., by incorporating sharp point(s), or edge(s). The cylindrical walls of the pressurizing member can incorporate one or more O-rings that can form a tight seal between the pressurizing member and the inside wall of the module, confining the sample within the interior compartment of the module. For some configurations, the pressurizing member can incorporate a handle or loop on the surface away from the sample to facilitate its insertion into or removal from the module. The pressurizing member can be designed to move freely within the module body to transmit the high pressure to the interior during the homogenization process, and can incorporate a feature (e.g., a seal) to act as an effective barrier between the sample and pressurizing fluid.

The module body can be designed to allow the pressurizing member to drive the sample material through the shredder screen/porous barrier. The module body can be, for example, made of a rigid or semi-rigid material such as metal (e.g., titanium, stainless steel, or aluminum), plastic (e.g., thermoplastic such as polypropylene, p-phenylene sulfide, or glass reinforced PEI resin), glass, stone, or a ceramic material. The module body is designed in conjunction with the pressurizing member and the cap to maintain appropriate pressure within the module body during the homogenization process.

The free movement of the pressurizing member allows the hydrostatic pressure inside the compartment to be transmitted to the interior of the sample chamber. Pressure equilibrium can be maintained between the inside and outside of the module, and the module material itself can be stable to the small differential pressure between the interior and exterior of the homogenization module prior to the pressure equilibrium being reached. In that way, the integrity of the module can be maintained. The module is designed to accommodate the appropriate ratio of sample and capture fluid volume relative to the air space to allow fluid to move across the barrier with each pressure cycle.

The barrier (e.g., screen) can be designed to further support the homogenization of the sample material as it is forced through the specified orifices of the porous barrier. The design of the orifices as to the size and the number of holes can vary, depending on sample type, size and homogenization process requirements. The barrier, screen, can be made of metal (e.g., titanium, stainless steel, or aluminum), plastic (e.g., thermoplastic such as polypropylene, p-phenylene sulfide, or glass reinforced PEI resin), glass, stone, or a ceramic material appropriate to solubilize the sample. Additionally, the barrier can also include a filter. The point of contact on the barrier with sample can be designed to assist in the homogenization of the sample by incorporating appropriate surface features, such as sharp point(s) or edge(s), or orifice configuration. The barrier can also be any porous material including a solid matrix of sand, fine glass beads, carbon, and/or sintered metal. The barrier can be designed as an integral part of the module body or as an inserted component of the homogenization module between two compartments of the device. The cap can be designed such as to seal the module body sample capture compartment, assisting in maintaining pressure during the homogenization process and allowing access to the sample capture compartments following processing. The cap can be made of metal (e.g. titanium, stainless steel, or aluminum), plastic (e.g., thermoplastic such as polypropylene, p-phenylene sulfide, or glass reinforced PEI resin), glass, stone, or a ceramic material appropriate to solubilize the sample. The cap can incorporate a feature (e.g., a helical ridge of a screw) to hold the pressurizing member within the module body. Above the top pressurizing member, an upper cap can also be used. It can be accessible to the pressurizing fluid, but can be designed so as to ensure that the pressurizing member is retained within the module during pressure cycling.

The entire module, optionally including a capture fluid, can be pre-assembled by a manufacturer, such that a user would only need to load the specimen, put on the top pressurizing member, and insert the unit into the pressure chamber. In the current configuration, the device is removed from the pressure chamber following PCT treatment. The pressurizing member on the side where the tissue is originally placed can then be pushed all the way against the barrier with the tissue side on the bottom, in opposite orientation relative to that when the tissue was first loaded. The cap (and bottom pressurizing member, if used) can be removed and the solution can be pipetted out. The cap can also be designed to incorporate a valve through which the solution can be allowed to drip out into a collection tube for minimum handling and direct transfer of material. Alternatively, instead of a cap, the device can be fitted with a puncturable membrane, such as an ethylene vinyl acetate (EVA) material on that used in a microtiter plate sealer. The membrane can be held in place by a cap, fused to the device wall with heat or radio frequency, or otherwise attached through adhesive or other mechanisms. The pressure across the membrane will be minimized by limiting the travel of the pressurizing member, which will result in pressure equilibration between the inside and outside of the device. Following PCT treatment, the device can be positioned over a collection tube containing a sharp puncture device which can break open the membrane and release the resulting fluid into the collecting chamber.

In addition to cells and tissues, fluids (e.g., sputum, mucus, or food products such as honey, molasses, or corn syrup), can also be processed in the devices. The device can be used to reduce the viscosity of such fluids or to liquefy samples so as to, for example, release microorganisms into a buffer or medium, with or without killing the organisms. Sputum, for example, which is generally liquefied using chemicals, can be liquefied in the devices of the invention, using pressure to release microorganisms such as Mycobacteria tuberculosis for further analysis or other treatment.

Part 2. The Processing Module

The new sample processing devices can also, optionally, include additional chambers for further processing of the sample. For example, the device can take the form of or include a “processing module.” While the design for such a processing module is typically the same as or very similar to the design for the homogenization module, further processing of the sample can be achieved in the processing module through passage of the sample through additional barriers into additional chambers. For example, instead of having only two chambers, as described in the homogenization module, the processing module can have as many chambers separated by penetrable barriers as required for the desired processing or analysis of the homogenized sample. The additional chambers of the Processing Module that are used to process the sample are typically separated by penetrable barriers, rather than the porous barriers used in the homogenization process. Separation of the processing chambers can allow reagents contained within the chambers to remain unreacted prior to introduction of sample.

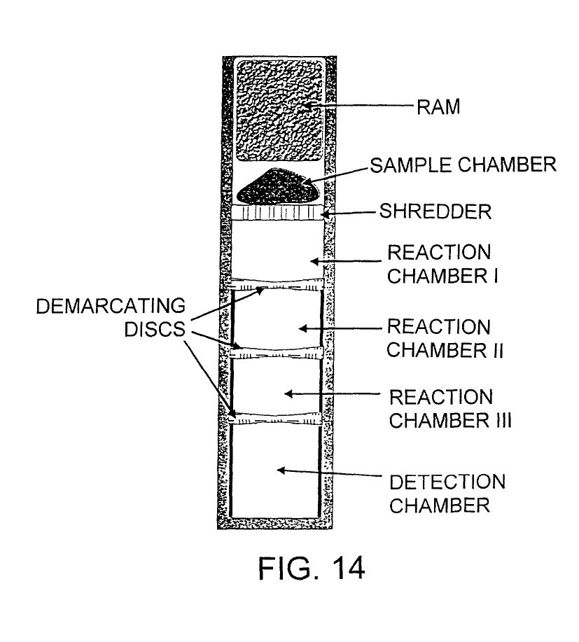

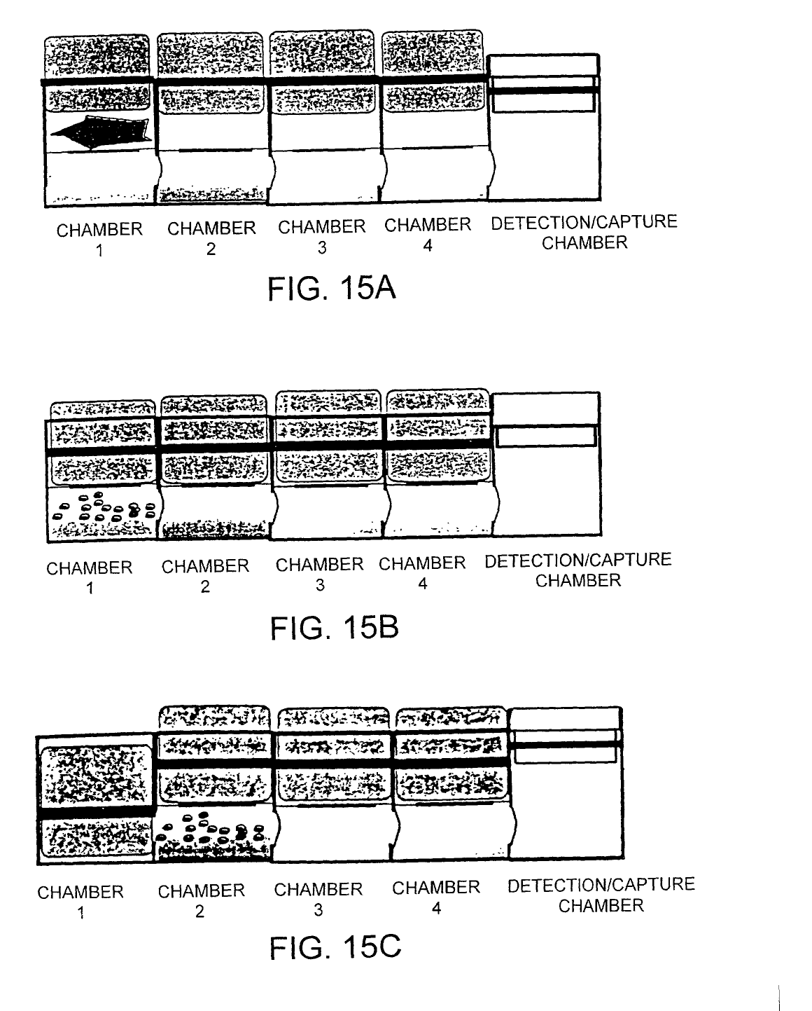

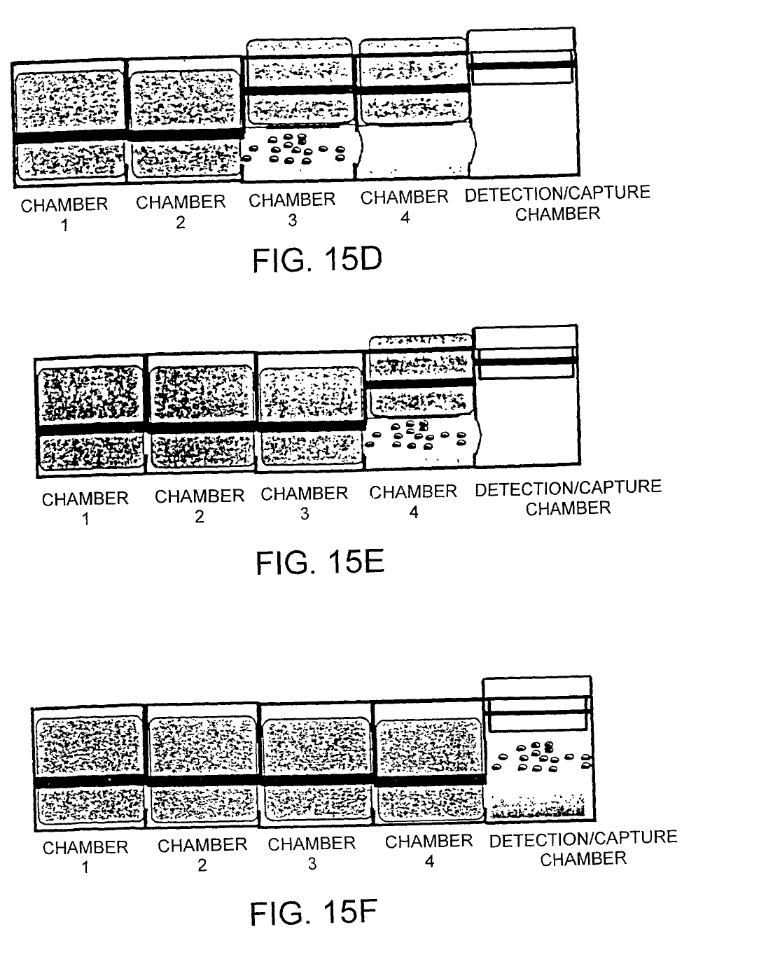

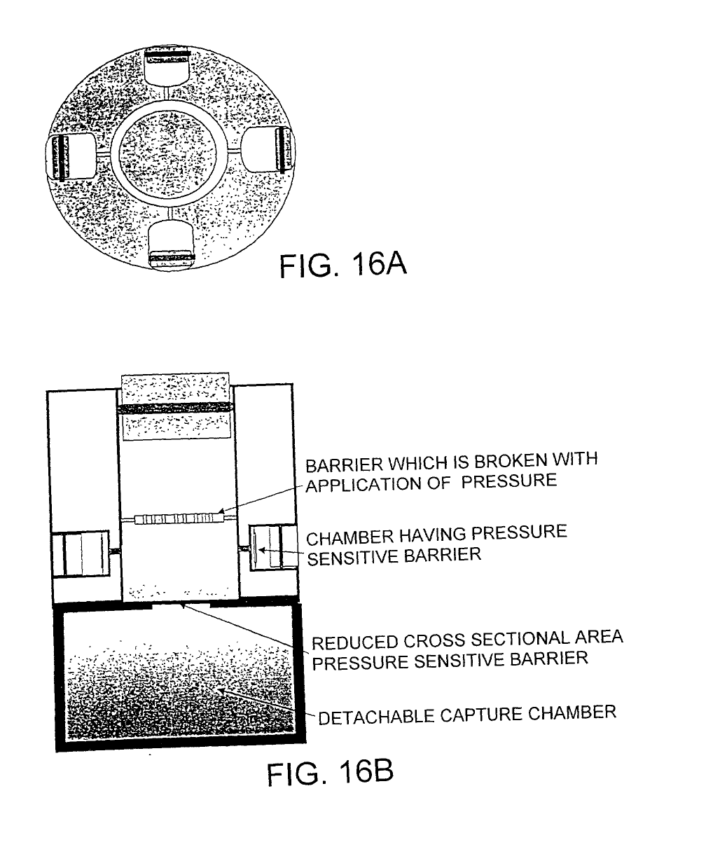

FIG. 14 illustrates a processing module wherein the chambers are positioned vertically. FIG. 15 illustrates a processing module wherein the chambers are positioned horizontally. FIG. 16 illustrates a processing module wherein the chambers are positioned both vertically and radially, separated by pressure sensitive barriers.

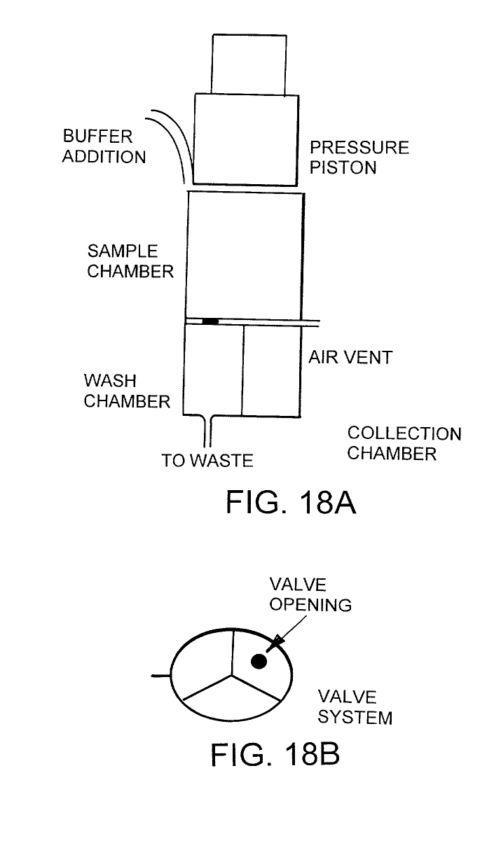

FIG. 18 illustrates a processing module wherein the chambers are positioned vertically, wherein the lower chambers can move radially with respect to the upper chamber and the barrier, allowing the sample to be separated from waste, debris and impurities from the processing. The upper chamber can be removed to retrieve the desired product (e.g., purified nucleic acid). All operations of the processing module can be automated and programmable by computer. Furthermore, the entire device is disposable.

The barriers can be penetrated under a variety of conditions, not limited to high pressure. For example, the barriers can be temperature sensitive, breaking down at a set temperature. The barriers can also be penetrated by exposure to solvents, causing the barrier to dissolve into solution. Additionally, the barriers can be valves, removed either mechanically or electronically.

Once the barrier is penetrated, the pressure from the pressurizing member forces the sample into the adjacent chamber, allowing the sample to interact with the reagents contained within the chamber. This process of forcing sample into subsequent chambers continues, allowing stepwise processing of the sample, and subsequent analysis if desired. The nature of the compartments within the device allows processing of the sample without manual handling.

Part 3. The Chamber Reagents

The identity of the reagents in each chamber can vary according to the processing to be achieved within that chamber.

For example, the physical and chemical properties of the reagents used in the capture fluid during the homogenization step can have an impact on the homogenization efficiency of pressure cycling and can also maintain the integrity and stability of the molecular components. As mentioned above, one possible homogenization mechanism is a “freezing and thawing” effect at subzero temperatures, and that effect will depend on the characteristics of the capture fluid. Secondly, the composition of the capture fluid has an impact on the release and dissolution of the biomolecules of interest. Thirdly, the volume of the capture fluid is important, since it relates to the maintenance of the module integrity, e.g., by providing resistance to excessive pressure from the pressurizing member. Lastly, the capture fluid needs to be compatible with the downstream assays.

Examples of the capture fluid can be found in the “Examples” section. In its simplest embodiment, the lysis fluid can be a hypotonic solution, such as water, or a low salt Tris or Phosphate buffer that promotes the dissolution of the cellular material out of the cells. Alternatively, the lysis solution can contain preservatives or chemicals that are compatible with downstream assays. Capture solutions can also contain enzymes to assist in sample disruption. Alternatively, nuclease or protease inhibitors can be added to the capture solution to preserve the integrity of the proteins and nucleic acids. For applications requiring the extraction of ribonucleic acids, denaturants (e.g., guanidinium salt and urea) or detergents (e.g., SDS and CHAPS) can also be added.

The reagents used in chambers subsequent to homogenization of the sample include a variety wide enough to allow a range of processing steps. For example, a compartment can contain a capture element, allowing separation of particular elements of the sample such as nucleic acids or hydrophobic peptides. Separation and/or purification of the sample can further be achieved through chromatographic reagents, or even an electrical current within a chamber.

One or more chambers can optionally contain growth and/or transport media, allowing for inoculation with organisms, which can then be incubated directly in the devices of the invention. A non-limiting list of such organisms includes cells, viruses, bacteria, or parasites. For example, blood cells and other animal or plant cells, HIV, HAV, HBV, HCV, Bacillus anthrasis, Mycobacteria tuberculosis, Vibrio cholera, Yersinia pestis, Salmonella, Shigella, Listeria, and Plasmodium species can be grown in the devices. The media can be liquid or solid, and can be specific for a given organism or non-specific. Examples of suitable media include sheep blood agar (SBA), trypticase soy agar (TSA), or trypticase soy broth (TSB) (e.g., for growth of Bacillus anthrasis); MacConkey agar (e.g., for growth of Gram negative bacteria); or triple sugar iron (TSI) broth. The transport media can be, for example, a buffered solution such as Tris-EDTA (TE). The devices can be incubated at an appropriate temperature for the given organisms (e.g., 35-37° C. for Bacillus anthrasis) for a sufficient time to allow for a detectable population, or to afford sufficient DNA for amplification by PCR or amplification of RNA by RT-PCR (e.g., followed by detection) or other methods. The growing organism can then be processed as described above (and optionally rendered inert) directly without opening the device, limiting the chances of contamination of the sample or the surroundings.

The chambers can contain elements that allow DNA hybridization following nucleic acid extraction. For example, the DNA can hybridize by competitive binding to different fluorescent labeled oligonucleotides contained in the compartment. Pressure can be subsequently applied to enhance the binding or dissociation of the hybridized oligonucleotides, as described, for example, in U.S. Pat. No. 6,258,534.

Reagents can include those required for amplification of nucleic acid sequences, for example, those used in polymerase chain reaction (PCR), ligase chain reaction (LCR), or reverse-transcriptase polymerase chain reaction (RT-PCR). Pressure cycling in the chamber containing these reagents can also be used to enhance these reaction. Temperature cycling can also be incorporated with these reaction conditions.

DNA sequencing can be achieved in one or more chambers having the required reagents. Again, cycled pressure (e.g., PCT) can be used to enhance this reaction. For example, high pressure can modulate exonuclease activity, dissecting nucleotide polymers into nucleotide monomers.

The reagents in the processing chambers can also be used to process proteins in a sample. Following the release of the proteins through cell lysis, proteins can be purified from the sample using such techniques as column chromatography or gel electrophoresis. Purification can occur based on molecular weight, or other physical or chemical properties such as size, hydrophobicity, ionic interactions, metal chelating properties, and immunological reactivities. Pressure and cycled pressure can be an aspect of this processing.

Additionally, the chambers can contain reagents involved in immuno- or enzymatic assays. For example, the association and/or dissociation of molecules can be controlled through the conditions within the chamber to control the specificity and affinity of specific complexes. The technology is applicable to both enzyme interactions as well as immunological interactions.

Part 4. Detection Module

One of the chambers can be used as a detection module for capturing a signal generated by the products as a result of the various processing steps applied to the sample. The detection module can be placed adjacent to a detector in such a way that the signal generated inside this module can be read by the detector. The module can be made of transparent material that permits light or radiation to pass through to the Detector. A laser or other ionization generator can also be placed opposite the detector to excite signal generation inside the detection module. The Detector can be a luminometer, fluorometer, photometer, spectrophotometer, ionization detector, flow counter, scintillation counter, camera or other analyzer. Signal received from the detector can be transmitted to a recorder physically or electronically to generate a measurement of the signal generated in response to specific analytes being measured in the sample. These measurements can be recorded in a computer, by print out, visually or by other means.

Part 5. Barriers, which are Shredders for Difficult-to-Macerate Samples

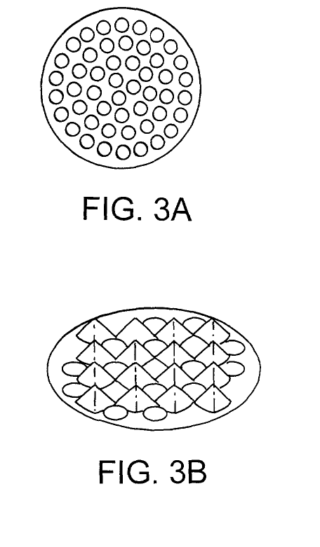

Very hard-to-macerate samples, such as animal bones, teeth, plant stems, and roots, often require special homogenization equipment, such as a pulverizer or anvil. One approach to homogenizing hard samples is to use in a device of the present invention a barrier that is a shredder screen of a more rigid material such as steel or high quality plastic. In some cases, a metal disk having holes through it can be used to reinforce an ordinary plastic lysis disk. In addition to using a barrier made of rigid material, simulated anvil type structures or pyramids made of rigid solid material will be incorporated into the barrier (see, e.g., FIG. 3B). Besides using a stronger module, pressurizing member(s) and barrier which is a shredder screen, a capture fluid of very low pH, such as pH 2, can be used to soften the bone or tooth. Because of its corrosive nature, such fluids are normally difficult to handle by manual processes, but can be readily accommodated in the closed system as described for PCT homogenization. Still further maceration of the sample can be accomplished, in the event maceration is incomplete, by using smaller samples. However, this can result in lowered sensitivity.

Part 6. Alternative Formats of Shredder for Lysing Biological Samples

The PCT homogenization principle incorporates two features which can promote tissue disruption and release of cellular content: the physical forcing of tissue material through small openings in the devices can contribute to breaking up large chunk of tissue, and the low temperatures and cycling pressure can subject the sample to repeated freeze-thaw thus further contributing to cellular disruption. The alternative formats of the devices can be designed based upon one or both of these principles. Various configurations of the module are envisioned, which can incorporate various module sizes and materials, and can include features for preventing collapse of the module, preventing fluid leakage, increasing the homogenization efficiency, providing easy accessibility to the sample, facilitating multi-sample processing, providing easy insertion and removal of the module to and from the pressure chamber, and ensuring containment of infectious material.

Part 7. Disposable Design for a 96-well Plate

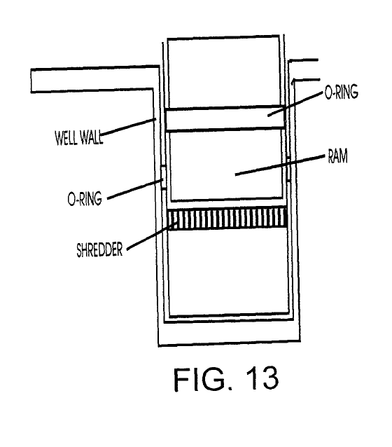

The device can be scaled up or down, thereby changing the corresponding sample size, compartment size, and reagent volume. Additionally, the scale of the module and chamber can be adjusted to accommodate multiple samples. The cap design for multiple samples can be different from that of the single device. The simplest configuration would be in the form of a sheet of sealable and/or peelable membranes that are flexible enough to stretch into the space vacated during the compression without breaking. A series of pressurizing members joined together on a rigid sheet could be pushed simultaneously into the individual wells, such as in a 96-well formatTo fit 96 samples into the pressure cycling instrument, the shredders can be arranged in an orderly array such as six 4×4 or three 4×8 blocks, which can fit easily into the pressure chamber and download directly into wells compatible with a 96-well format for subsequent processing, if desired. (See FIG. 13)

FIG. 13 illustrates a pressure cycling instrument that fits into one well of a 96 wells plate. The barrier divides the two chambers, and the pressurizing member is sealed by an O-ring between the pressurizing member and the chamber wall. The sample is placed on tope of the barrier and will be pushed through the barrier by the pressurizing member. When the pressure cycling is complete, the barrier and pressurizing member will be removed and the solution in the second chamber can be transfered for further processing. This chamber can be fitted to a pressure cycling reactor such as that described in U.S. Pat. No. 6,036,923.

The device can also be designed such that the screen and pressurizing member are removable as a single unit after completion of the process.

For processing larger numbers of samples, a 96-well system can be used, in which samples are first lysed and subsequently transferred to another chamber or well for washing and collection of filtrates. Alternatively, a disposable microwell format with multiple chambers can be used to handle the entire extraction process. In this configuration, an initial chamber containing the sample can be subjected to pressure lysis. A valve in the well would then open to allow washing of the sample and carrying away the waste. Finally, another valve leading to the nucleic acid collection chamber would allow elution of the purified nucleic acid. Ideally, the entire process can be accomplished in a single purification module with minimum fluid exchange.

A simplified schematic diagram of the components of this device is shown in FIGS. 15A-15F. FIG. 15A illustrates the multichamber device wherein the sample is loaded into the upper half of the first chamber, but no pressure has been applied. FIG. 15B illustrates the result of initial application of pressure to the multichamber device, wherein the sample has been processed and has been pushed through the first barrier. FIG. 15C illustrates the result of a further increase in pressure, wherein the sample has been pushed horizontally into Chamber 2. FIGS. 15C-15F further illustrate the movement of the sample through the chambers, which are connected together in a horizontal manner, as pressure is increased. This device preferably does not require highly skilled technicians, or manual handling of toxic or hazardous chemicals. The purified nucleic acid products can be made available in a 96-well configuration, which can be compatible with other automated instruments for amplification, sequencing or other analysis.

Part 7. On-Chip Homogenization by PCT Process

The homogenization process can be further miniaturized to incorporate multiple functionalities for specific laboratory applications, such as nucleic acid or antigen detection. The shredder screen, pressurizing member, capture fluid, and holder can be made part of an integrated disposable unit that can deposit the extracted material to another location on a chip upon finishing the homogenization process. The chip can then be inserted into the analyzer and the temperature equilibrated around the sample homogenization area using a heat sink. Pressure can be transmitted via two pistons, one from the pressurizing member side and the other from the cap side. The pressure process is similar to the single shredder operation. Pressure can also be generated by electrical magnetic pistons, rather than by using conventional hydraulic pumps. One advantage of using electrical magnetic pistons is that they can be moved at higher frequencies, such as on the millisecond scale. As soon as the pressure process is finished, the capture fluid and/or the PCT processed sample, can be withdrawn from the “cap” side by, for example, “piercing the cap.”

Part 8. Disposable Device Design and Fabrication

The device can be a disposable module, made of, for example, polymeric material, while maintaining its suitability for sample preparation and for lysis of individual cells and tissues.

### EXAMPLES

The invention is further described in the following examples, which do not limit the scope of the invention described in the claims.

### Example 1

### Lysis of Rat Liver Sample using Cycled Pressure at Low Temperature

Whole pieces of fresh frozen rat liver, which were immediately frozen on dry ice, and kept either on dry ice or at −70° C., were obtained from Pel-Freez (Rogers, AR). The frozen tissue was cut on a block of dry ice using a razor blade to approximately 0.20 g, as determined by weighing on an electronic scale. The sample was kept frozen during the cutting procedure and stored in microcentrifuge tubes at −70° C. until use.

The device used in this experiment was comprised of a body, two rams, and a shredder stage made of polypropylene (see FIG. 2). The body was a cylindrical tube, 38.1 mm long, with an outside diameter of 13.8 mm, and an inside diameter of 11.6 mm. One end of the tube was threaded to accept a cap. The cap was used to seal and hold the ram at the end of the tube. The rams were cylindrical shaped parts that were 12.7 mm long and 11.5 mm diameter. Each of the rams was equipped with an O-ring seal that sealed against the interior surface of the body. The shredder stage was a cylindrical tube in shape, 9.53 mm long, and with an outside diameter of 11.5 mm, and an inside diameter of 10.7 mm, with the screen attached at one end. The screen had 49 holes with diameters of 0.94 mm appropriately spaced in the 11.5 mm diameter (see FIG. 3A).

The device was assembled by, first putting the bottom ram into the body at the threaded end, and then tightening the cap to hold the bottom ram on the body. 0.7 ml capture fluid (saturated guanidinium HCl, 1% CHAPS; saturated GTC, 0.1% NP40, or a proprietary lysing solution) was loaded from the open end. Then, the barrier, which was a shredder stage, was placed inside the body with the end with the holes away from the threaded end. A piece of frozen tissue, approximately 0.20 g was placed into the body atop the shredder stage. Once the sample had been positioned atop the shredder stage, the top ram was inserted into the body, with the seal ring creating a seal with the body.

For pressure cycling, the module was placed into pressure chamber (BaroCycler™ V2.4, custom built by BBI Source Scientific, Garden Grove, Calif.) that had been pre-chilled and equilibrated to −20° C. using a Neslab circulating chiller. The shredder with sample was equilibrated for temperature for 2 minutes. Pressure was applied to the sample at 15 psi, held at that pressure for 20 seconds, and then brought back to atmospheric pressure for 20 seconds. The pressure ramp up and ramp down times were less than 10 seconds and were not included in the hold times described. This sequence was repeated again for a total of 5 cycles. Following these 15 kpsi pressure cycles, an additional three pressure cycles of 20 seconds at 35,000 psi and 20 seconds at atmospheric pressure were applied. All the pressure cycles were programmed and carried out automatically by executing the program. Upon finishing the last pressure cycle, the shredder module was taken out of the reaction chamber and turned upside down. The cap that was originally on the bottom and the adjacent ram underneath were removed and the capture fluid including the tissue debris was transferred to a new microcentrifuge tube. The sample was centrifuged at 7,400× g for 1 min, and the supernatant was transferred to a new tube and was kept on dry ice.

A portion of the crude supernatant was also purified by the Roche Molecular Biochemicals (Indianapolis, Ind.) PCR template purification kit to extract the released nucleic acids in the supernatant. The procedure as described in the kit was followed, except that the lysis step using proteinase K was omitted. 200 μl sample was mixed with 200 μl binding buffer provided with the kit, 140 μl double distilled water and 100 ml isopropanol. The solution was then mixed by vortex and transferred to the high pure filter tube and centrifuged at 8,000 rpm for 1 min. After centrifugation, the filter was washed with 500 μl washing buffer by centrifugation again at 8,000 rpm for 1 min. After the first wash, the wash step was repeated and centrifuged. A 10 second centrifugation at 13,000 rpm was applied to remove residual wash buffer. To elute the nucleic acids, 200 μL pre-warmed (70° C.) elution buffer was added to the filter tube and centrifuged at 8,000 rpm for 1 min.

To estimate the yield and size of the nucleic acid released by the PCT homogenization procedure, an agarose gel analysis was performed using 0.67% agarose in 0.5′ TBE buffer, running at constant 120 V for 60 minutes. The agarose gel was pre-stained with ethidium bromide and photographed using a Chemilmager™ and AlphaEase™ V5.5 software (Alpha Innotech Corp, San Leandro, Calif.)

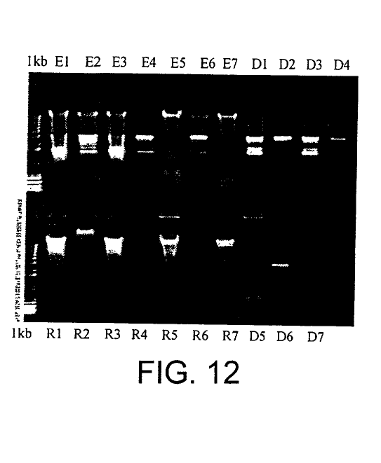

FIG. 12 illustrates the total nucleic acids present in the crude lysate (Lane E1-E7), without carrying out any purification procedures, where samples 1 (with sat. guanidinium.HCl, 1%CHAPS), 3 (with sat. GTC, 1%NP40) and 5 (with proprietary lysing buffer) were obtained by PCT tissue shredder treatment, cycled 5 times at 2×100 MPa and 3×235 MPa, −25° C., 20 s high pressure/20 s 1 atm holdings. Samples 2, 4 and 6 were no-pressure controls for sample 1, 3 and 5 respectively. Sample 7 was mortar/pestle homogenized. Also on FIG. 12, Lane D1-D7 and R1-R7 were samples extracted with Roche High Pure PCR template kit and treated with DNase (D1-D7) or RNases (R1-R7). The results showed that the PCT treated sample achieved similar levels of genomic DNA as the Roche Molecular kit, judged by the band intensity of the agarose gel (FIG. 12, lane R1, R3, R5, and R7) or O.D. 260 nm reading. Negative controls were obtained following the same treatment except pressure is kept at atmospheric level during the experiment.

As shown in FIG. 12, large amounts of genomic DNA (gDNA; lanes R1, R3) and ribosomal RNA (rRNA; lanes D1, D3) were preserved and released by PCT shredder as compared to no-pressure control (Lane D2, R2, D4, R4). The yield of gDNA was actually greater than that of the positive control obtained using the Roche kit procedure, which included a 4-hour pre-incubation step with proteinase K to solubilize the tissue prior to extraction (Lane R2, R4 versus R7) in this experiment.

### Example 2

### Study of Tissue Lysis Efficiency and Improvements of The Two Chamber Homogenization Module Design

Using the rat liver as a model, tests were performed on pressure profile (from 15 to 50 kpsi), cycle number profile (from 2 to 45 cycles), temperature profile (from −30 to 0° C.), number of shredder orifices (from 10 to 89 holes on identical area) and a variety of capture fluids (Gdn/1% CHAPS; phosphate buffered saline, GTC/1% NP40). Following each test condition, the capture fluid was transferred to a new microcentrifuge tube and briefly centrifuged at 7,600× g. The supernatant was collected and stored in new microcentrifuge tubes. Nucleic acids were extracted from the supernatant using commercially available kits. For analysis of DNA yields, purified nucleic acids were subjected to RNase treatment followed by analysis by agarose gel electrophoresis. The recovered material was treated with RNase prior to electrophoresis in a 0.8% agarose gel in 0.5× TBE buffer, running at constant 120 V for 60 min.

FIG. 4 illustrates one of the pressure cycling profiles. The yield of gDNA released by 5 cycles of PCT (Lane 3) was greater than that released by 2 cycles of PCT (Lane 2) as determined by integration of the densitometric scan of the band intensities for gDNA on the gel and was comparable to that obtained by the control method using the Roche kit. Similarly treated supernatant from tissue held at −25° C. and at atmospheric pressure is shown as a negative control (Lane 1).

These results demonstrated at least an equivalent release of nucleic acids from tissues by the two chamber homogenization module method of treatment as achieved using the standard methods, without the requirement for extensive (4 hr) proteinase K digesting. The yield of genomic DNA was similar to that obtained by a positive control. The RNA was well preserved judging by the ribosomal RNA bands. The relatively high background of nucleic acids seen in one of the no-pressure controls, sample 2 of FIG. 12 was not typical for these tissue samples and was not seen in other experiments. Lane D7 has no RNA bands, since during the prolonged incubation step with proteinase K specified in the Roche kit procedure, endogenous RNases effectively degraded any RNA released. The PCT shredder itself used in these studies appeared to be relatively pressure-stable and reusable after cleaning. Unlike sealed tubes made of similar plastic as used in the shredder and subjected to PCT conditions, no stress marks were observed on these modules, presumably because the pressure inside and outside the modules was readily equilibrated by the movement of the ram.

### Example 3

### PCT homogenization of Rat Brain, Pancreas, Large Intestine and Skeleton Muscle

The effectiveness of PCT homogenization was also evaluated on several additional tissues, which present distinctive difficulties in extractions. Brain tissue is particularly rich in proteins and lipids, which usually present white, flocculent material in the aqueous phase during an aqueous-organic liquid phase extraction. Pancreas is extremely high in nuclease concentration, such that RNA is usually degraded and difficult to recover by conventional methods. Large intestine and skeleton muscle are fibrous tissues with connective tissue components, making them difficult to homogenize.

These tissues were PCT processed under the same experimental procedure in the liver used for FIG. 12, except 100 μl, instead of 200 μl lysate was extracted using the Roche High Pure PCR template kit. The final elution volume was 60 μl. The gDNA and rRNA were determined by an agarose gel. FIG. 5 shows that the homogenization efficiencies of the PCT process on intestine and muscle were comparable to the conventional proteinase K method. Lanes 1-5 contained genomic DNA released from large intestine. Lanes 6-10 contained genomic DNA released from skeleton muscle. Lanes 1, 3, 6, and 8 were samples homogenized using the PCT module. Lanes 2, 4, 7, and 9 were no-pressure controls respective to lanes 1, 3, 6, and 8. Lanes 5 and 10 were positive controls by proteinase K homogenization. Lanes 1, 2, 6, and 7 were processed in the presence of sat. Gdri/1% CHAPS. Lanes 3, 4, 8, and 9 were processed with 6M GTC/1% NP40. All samples, except the no pressure control were supernatants from the crude lysate and extracted with Roche High Pure PCR template kit without the proteinase K treatment.

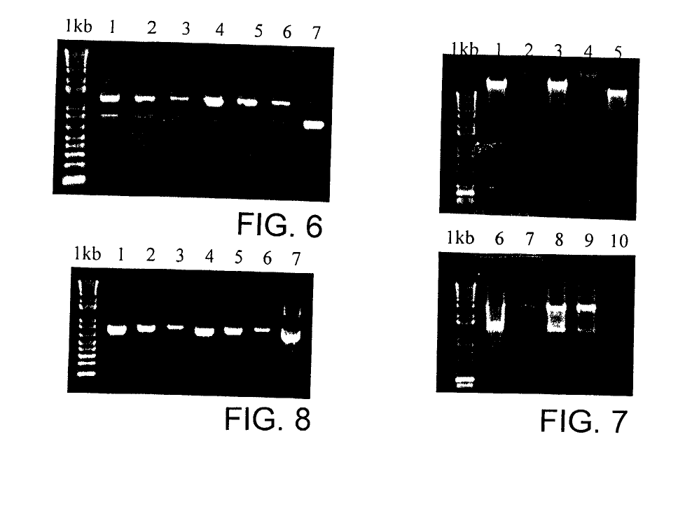

The genomic DNA bands presented in the PCT lysate were amplifiable and resulted in identical PCR products produced by the positive control (FIG. 6). FIG. 6 illustrates PCR amplification of genomic DNA from skeleton muscle samples using P-actin primer set from Clontech. Lanes 1, 2, and 3 were PCR products from 1:5, 1:25 and 1:625 diluted PCT treated template. Lanes 4, 5, and 6 were PCR products from 1:5, 1:25 and 1:625 diluted ‘positive control’ template. Lane 7 illustrates the PCR product using β-actin cDNA control obtained from Clontech.

FIG. 7 illustrates rate brain lysate extract. Lanes 1, 2, 6, and 7 were processed in the presence of saturated Gdn/1% CHAPS. Lanes 1 and 6 were from a PCT homogenized sample. Lanes 2 and 7 were sample from no-pressure control. Lanes 3, 4, 8, and 9 were treated with 6M GTC/1% NP40. Lanes 3 and 8 were from pressure treated sample, wherein lanes 4 and 9 were no pressure controls. Lanes 5 and 10 were from the Roche kit ‘positive control.’ Lanes 1-5 were treated with RNase. Lanes 6-10 were treated with DNase. Sample 10 lacked RNA due to the proteinase K step.

RT-PCR RNA templates from PCT-treated or no-pressure treated tissue supernatants were obtained by extraction with the Roche High Pure PCR template kit procedure, but omitting the proteinase K pre-digestion step. The “positive control” was generated from an identical tissue specimen using the Roche High Pure Tissue™ RNA procedure including the proteinase K digestion step. The resulting extracts were subjected to RT-PCR using the QIAGEN® One Step RT-PCR protocol and primers for β-actin obtained from Clontech (Palo Alto, Calif.). To estimate the yield of nucleic acid released by the PCT homogenization procedure, an agarose gel analysis was performed using 0.8% agarose. Results show that RT-PCR products recovered from PCT templates were comparable to the kit control. Interestingly, it seems that more mRNA were recovered from the pancreas sample by the PCT method than by the control Roche kit. FIG. 8 illustrates P-Actin RT-PCR products from rat brain templates. Lanes 1-3 were from a pressure extracted sample. Lanes 4-6 were from a positive control sample prepared using Roch High Pure tissue RNA kit. Template dilution factors for 1-3 and 4-6 were 1:5, 1:25 and 1:625 respectively. Lane 7 contained a Clontech primer set control.

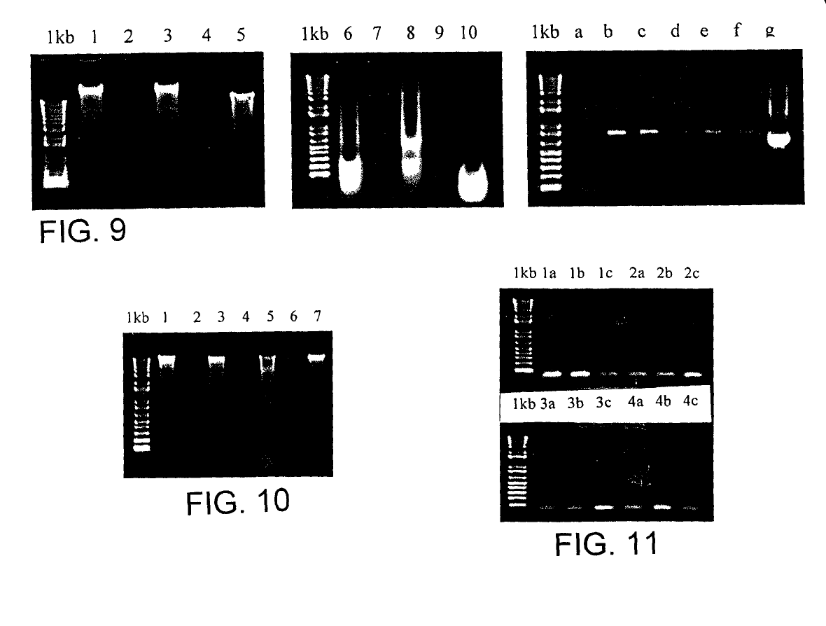

FIG. 9 illustrates rate pancreas homogenized by the PCT module. Lanes 1-5 showed genomic DNA in the extract and lanes 6-10 showed rRNA from the same extracts purified using Roche High Pure Tissue RNA kit. Samples were treated similarly to those for the rat brain shown in FIG. 7 Lanes a-g from the pancreas were equivalent to lanes 1-7 described in FIG. 8 from rat brain above.

The lack of signal in Lane a, FIG. 9 was likely due to the existence of PCR inhibitors in the template. This experiment was repeated and the data showed similar results. The most important observation is that the PCT method is effective in releasing and protecting mRNA from pancreas, a particularly difficult task to accomplish by conventional tissue homogenization methods.

### Example 4

### Plant Tissue Homogenization: Corn Leaf Nucleic Acid Release and PCR Amplification

Additional experiments were conducted to demonstrate homogenization and release of genomic DNA, ribosomal and messenger RNA from corn leaves. The standard method of release of nucleic acids from corn leaf requires mincing with razor blades followed by mortar and pestle homogenization. In order to preserve nucleic acids from degradation, the homogenization process is typically performed in the presence of liquid nitrogen. The tissue is then incubated with proteinase K for release of nucleic acids followed by extraction for genomic DNA using standard extraction methods or kits.

In the PCT homogenization experiment, a fresh corn leaf was collected after germination for 7 days. 0.2 g of fresh leaves was rinsed with cold distilled water and temporally stored in microcentrifuge tubes at −70° C. For these studies, 0.7 ml extraction buffer (6M guanidinium/1% CHAPS; 10 mM Tris.Cl, pH 8.0, 10 mM EDTA [TE]; or GTC/1% NP40) was added into the capture compartment, of the shredder module and subjected to PCT treatment as described for the animal tissues. The pressure pulsing sequence is similar to that described in Example 1. After the pressure treatment, capture fluid was collected and briefly centrifuged, 8,400× g for 1 min, to remove debris. The supernatant is collected and analyzed by an agarose gel electrophoresis.

FIG. 10 illustrates the release of DNA using the PCT module. Lanes 1 and 2 show samples processed in a 6M Gdn/1% CHAPS buffer. Lanes 3 and 4 were processed in a 10 mM Tris.Cl, pH 8.0, 10 mM EDTA [TE] buffer. And, lanes 5 and 6 were processed in a GTC/1% NP40 buffer. No pressure control samples were shown in lanes 2, 4, and 6. A QIAGEN plant DNA extraction kit process was applied to the sample in lane 7. As shown in FIG. 10, the results illustrated that similar amounts of DNA were released from corn as compared to that by QIAGEN® DNeasy Plant Minim kit, which required extensive grinding with a mortar and pestle in liquid nitrogen prior to extraction (FIG. 10). Interestingly, even when TE buffer was used as collection solution, significant amount of DNA was recovered. All DNA samples served efficiently as templates for PCR reaction, giving similar results to those seen with the control template extracted with the mortar and pestle.

The results illustrated in these examples show that PCT treatment can be used to liberate nucleic acids form a variety of animal and plant tissues without the requirement for extensive digestions with proteinase K or the need for homogenization with mortar and pestle. Both RNA and DNA are recovered efficiently, with little degradation of RNA observed. Nucleic acids can be released by PCT even in buffers (e.g., TE) in the absence of detergents.

Crude lysates are suitable for use as templates in downstream processing, such as PCR. FIG. 11 illustrates PCR amplification of corn leaf DNA released from the above described process. The primer set amplified the ‘MTTC’ region in the corn genome. Lanes 1a-1c, 2a-2c, and 3a-3c contained PCT module released sample. Lanes 4a-4c contained the positive control sample. Lanes 1a, 2a, 3a, and 4a, contained template concentration of 1:5. Lanes 1b, 2b, 3b, and 4b, contained template concentration of 1:25. Lanes 1c, 2c, 3c, and 4c contained template concentrations of 1:625. The results demonstrate that DNA obtained using the PCT module yielded identical product to the ‘positive control.’ The methods described therefore are much faster and easier to conduct and result in high yields of good quality nucleic acids using fewer steps than conventional methods.

### Example 5

### Multichamber Device for Purification of Nucleic Acid by Pressure and Electric Current

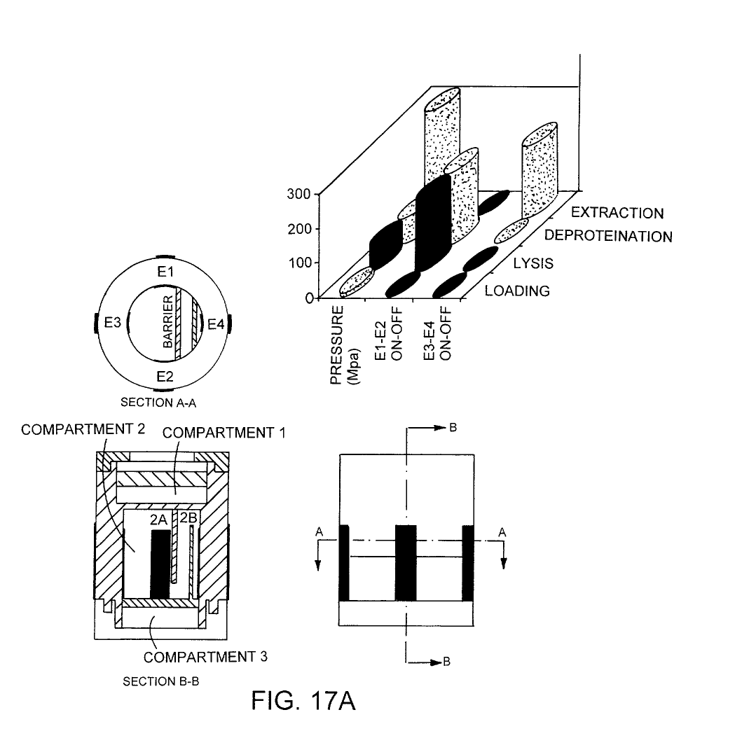

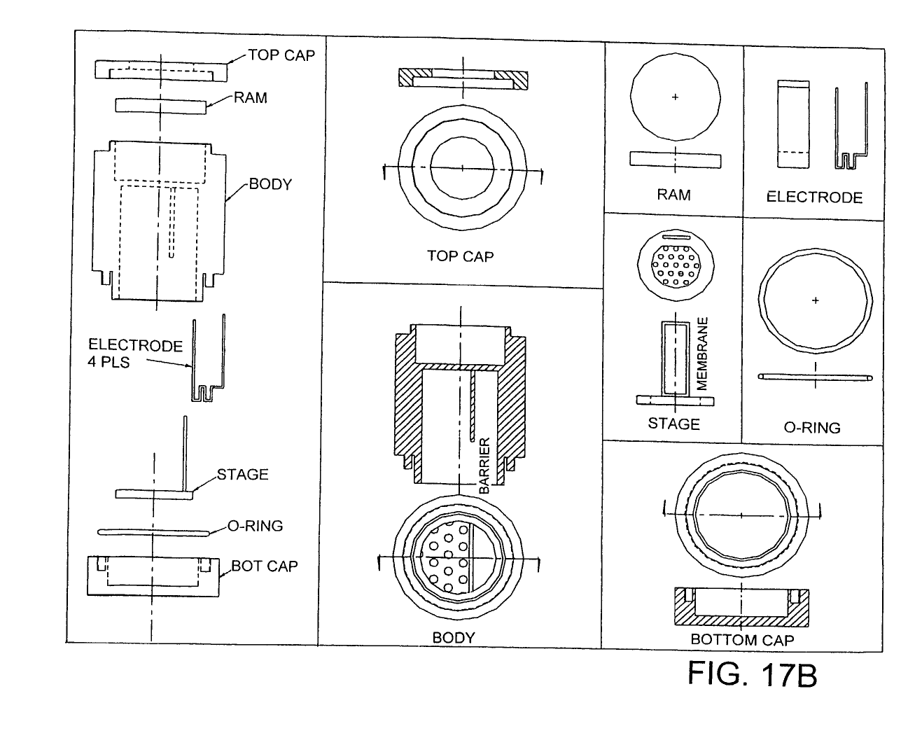

In one configuration, pressure can be combined with an electric field to release the nucleic acids from the cells and purify them (FIGS. 17A-17B). FIG. 17A illustrates 7 parts of the extraction module and assembly are shown (e.g., the top cap, the pressurizing member (a ram), the body of the module, an electrode, a barrier staged at the lower portion of the module, an sealing member (an o-ring), and a bottom cap). These features are also shown in cross section. FIG. 17B illustrates the function of the assembled module. The sample is placed into chamber 1, and the RAM is moved to deliver sample into chamber 1 during ‘Loading’ period. Between chamber 1 and 2, this device is also equipped with a barrier (e.g., a shredder) for solubilizing tissues. After applying cyclic pressure under appropriate temperature, lysis of cells and release of nucleic acids occur. Following cell lysis, the lysate is transferred into chamber 2 by passing the barrier (shredder) between the compartments via pressurization. Temperature can also be important to induce the pressure drive freeze-thaw effects. Following the ‘Loading’, ‘Lysis/Deproteination’ involving application of cycled high pressure begins. Electrode E1-E2 is on and pressure is cycled between 15 and 75 MPa. Then, the ‘Extraction’ is initiated at elevated pressure with electrodes E3-E4 activated. In chamber 2, the released nucleic acids would bind to a resin with specific affinity for nucleic acids. An electric current applied to the chamber results in migration of nucleic acids from the binding resin in chamber 2A to an electrode in chamber 2B (See FIG. 17B). The movement of nucleic acid from chamber 2A to chamber 2B introduces a chromatographic component to the nucleic acid isolation procedure, making it possible to separate nucleic acids of different sizes by varying the pressure and intensity of the electric field. Depending upon the nature of sample, this process may take 1-15 min. Following electrophoretic separation, the nucleic acids are eluted into a collection chamber, Chamber 3B, from which purified nucleic acids are subsequently removed by recovering the binding membrane. A high pressure instrument (e.g., BBI's Barocycler™) capable of supporting both the cell lysis and nucleic acid separation allows both high throughput processing and analysis. The reaction chamber can be designed to accommodate multiple modules.

### EXAMPLE 6

### Purification of Nucleic Acids by Pressure and Binding to Resin

A nucleic acid preparation module enables cell lysis and purification of nucleic acids by step-wise passage through a series of chambers (FIG. 18). This device consists of an upper sample chamber into which the sample is loaded and in which the cell lysis takes place. This chamber is sealed off from the outside pressurizing fluid by a ram having a rubber gasket. The chamber can also contain a porous support (for cells) or porous “shredder” to facilitate fragmentation of tissue chunks into smaller pieces to expedite nucleic acid extraction by forcing the tissue through these pores under pressure. The cap, or ram, serves both to seal the module and to transmit pressure to the sample. Following PCT (cycling of high pressure), the extraction buffer containing nucleic acids is be transferred from chamber 1 to chamber 2 for purification of the nucleic acids. Nucleic acid could be bound to a matrix, such as silica, ion exchange resin, or commercially available reagents such as QIAGEN® plasmid extraction, or QIAamp extraction columns. The transfer of liquid between compartments is mediated by a rupture of the barrier between compartments through application of pressure. Temperature or mechanical means can also be used. Alternatively, a series of valves and tubes can be incorporated into the multichamber device, which would enable the addition and removal of fluids as well as transfer of reagents from one chamber to the next.

Chamber 2 contains a DNA (or RNA) binding resin (e.g., silica DEAE) that binds nucleic acids. A liquid input/output system is used to introduce and remove liquids allowing the debris and impurities to be washed away into Chamber 2. The purified nucleic acid is then eluted into the collection chamber (Chamber 3). A valve directs the flow of wash buffer to the waste receptacle and the purified nucleic acid to the collection chamber.

In this configuration, sample is transferred into the chamber containing lysis buffer and binding matrix. The extraction module is sealed and inserted into a BaroCycler™. Following pressure treatment, debris is washed away at low pressure and DNA eluted into the collection chamber at high pressure. This flexible design allows selection of different lysing and washing buffers and nucleic acid binding matrices, and also allows programming of pressure conditions required for each subsequent processing step.

The extracted nucleic acid can be used directly for microarray analysis or amplification without further purification. Alternatively, the solute might be applied to one of the lab-on-chip systems with components for DNA sizing and separation or for the separation of total and mRNA. If necessary, DNA and/or RNA can be extracted from the total nucleic acid preparation using commercial extraction kits.

### Example 7

### Lysis in Low Salt

Achieving cell lysis in low salt solutions without detergents, allows the resulting nucleic acid to be sufficiently pure for many subsequent analytical procedures including PCR and sequencing. Impurities such as proteins and other debris can be bound to a different matrix, such as Chelex™, or Procipitate™ leaving the nucleic acid in solution. In this configuration, (See FIG. 18) the debris and impurities are retained in Chamber 1, while the soluble nucleic acid collected into Chamber 2 by simple filtration. The released nucleic acids are subsequently used directly in a variety of biochemical and enzymatic applications, including restriction endonuclease digestion, PCR, DNA sequencing and other analytical methodologies.

The devices described in this patent provide a complete, self-sustained system for nucleic acid purification, taking advantage of pressure mediated lysis and deproteination, followed by purification to obtain a concentrated nucleic acid product. Subsequent processing of the nucleic acid can include washing away the debris at low pressure and eluting the purified nucleic acid under high pressure in a second step, using a BBI BaroCycler™ v3.0.

### Example 8

### Double O-Ring Device

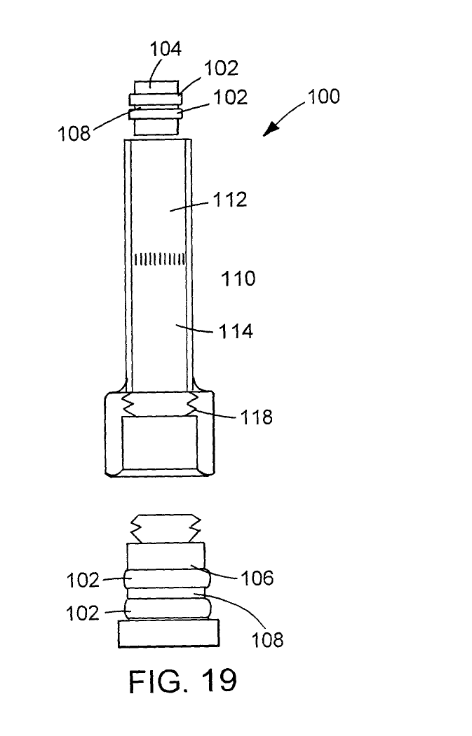

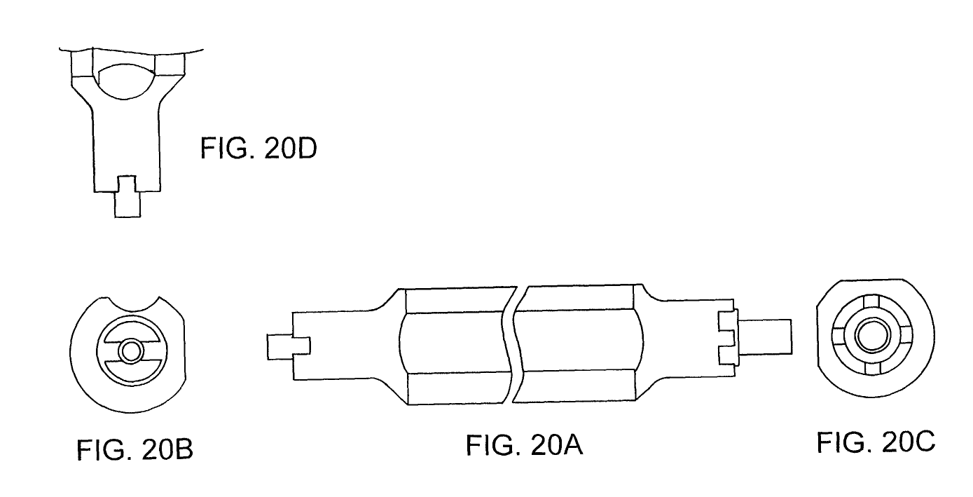

FIG. 19 is a drawing of a multichamber device 100 of the invention having double O-rings 102 on both a ram 104 and a screw cap 106. There are also grooves 108 in the ram and cap that can capture any fluid that may have gotten past either O-ring from inside the tube or from outside the tube. The device also includes a lysis disk 110, a sample chamber 112, and a fluid/reaction chamber 114. In a typical use of the device 100, fluid/reaction chamber 114 is charged with a buffer solution, screw cap 106 is screwed into a threaded portion 118 of device 100 adjacent to chamber 114, a sample to be processed or macerated is put into sample chamber 112, and ram 104 is inserted into the sample chamber, optionally using a tool that enables the ram to be inserted to a preset depth. An example of such a tool is shown in FIG. 20. One end of the tool shown in FIG. 20 acts as a screw driver to screw in or unscrew cap 106. The other end of the tool has a post that sets ram 104 to a predetermined proper depth. The device 100 is then put into a pressure cycling apparatus such as a BBI BaroCycler™, which causes the ram to force the sample through lysis disk 110, generally resulting in a solution or suspension of the lysed sample in the buffer solution in chamber 114.

It can be determined whether the devices 100 “leak” from either inside or outside of the tube by using a known concentration of fluorescein solution and measuring the fluorescence of the solution inside the tube or fluid in bags outside the tube. When the fluorescence is compared to a dose response curve made from fluorescein in the appropriate buffer, the amount of fluorescence can then be converted to volume. This method is very sensitive and can be used to detect leaks of as low as 0.1 μl volume. The method can be used for evaluation of newly made tubes, for quality control, or for samples that have been processed.

### Example 9

### Rat Tail Lysis by PCT

Rat tails were obtained from Pel-Freez Biologicals, freshly frozen and kept on dry ice or at −70° C. 0.2 g pieces of rat tail were processed at 35 kpsi, 4° C., for five one-minute cycles using a BBI Barocycler™ v2.4 in multichamber devices of the invention as shown in FIG. 19. The devices were each supplemented with a stainless steel disk having twenty 2 mm holes as shown in FIG. 3A. The purpose of the metal disks is to reinforce the plastic lysis disk in the body of the devices. Buffers used in this set of samples are listed in the third column of Table 1:

TABLE 1

| | | | | % of DNA |
|---|---|---|---|---|
| | | | | Yield by |
| | | | DNA | proteinase K |
| Sample | | | conc. | digestion |
| Number | Treatment | Lysis Buffer | (μg/mL) | method |
| 1 | Protease K 55° C. | Qiagen ATL buffer | 41 | 81% |
| | 30 min. + PCT | | | |
| 2 | Protease K 55° C. | Qiagen ATL buffer | 79 | 100% |
| | 5 hours | | | |
| 3 | PCT | Qiagen ATL buffer | 72 | 91% |
| 4 | PCT | Sat. Gdn./1% Chaps | 71 | 90% |
| 5 | Mortar & Pestle | Qiagen ATL buffer | 63 | 80% |
| 6 | Negative control | Sat. Gdn./1% Chaps | 2 | 3% |

After obtaining the crude lysate from the three different methods defined by the lysis buffers, the QIAGEN® QIAamp™ DNA Tissue kit protocol was followed to purify genomic DNA from the samples, except that the proteinase K treatment step was omitted for the “PCT” and “Mortar & Pestle” samples (i.e., samples 3-5). The negative control sample was obtained by treating the sample in the same way, except that no pressure treatment was conducted. The corresponding DNA yield indicated in Table 1 was obtained by O.D. 260 readings.

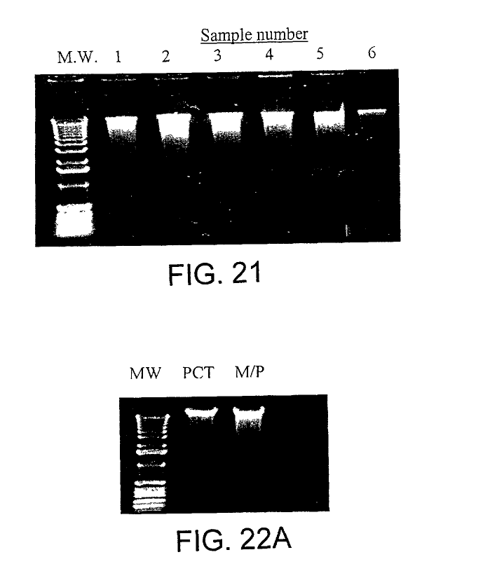

As demonstrated by agarose gel electrophoresis of the genomic DNA purified from samples 1-6, shown in FIG. 21, the lysis efficiency accomplished using the pressure cycling (“PCT”) is comparable to the conventional proteinase K digestion or mortar-pestle methods. Agarose gel shows listed in Table X. Same volume of purified DNA samples were loaded onto each lane. Other experiments (data not shown) demonstrated that similar amounts of total RNA were successfully extracted from the rat tails compared to the mortar & pestle method. The DNA and RNA yielded from the PCT treatment were PCR-amplifiable as exhibited by the PCR or RT-PCR using β-actin primer sets.

### Example 10

### Rat Brain Processing and Extraction of Molecules Therefrom

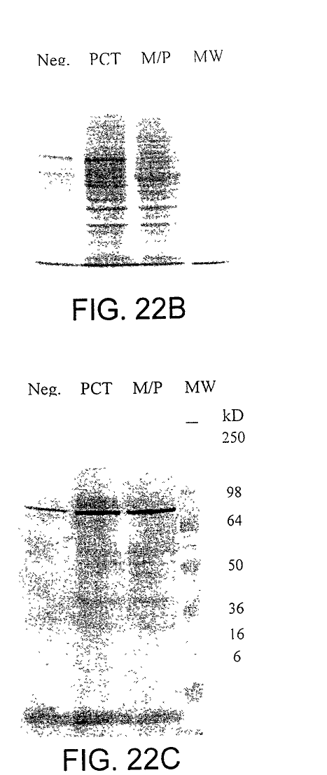

Rat brain samples were obtained from Pel-Freez Biologicals. The samples were fresh frozen, and then stored on dry ice or at −70° C. The samples were treated by PCT at 35,000 psi ° C., for five one-minute cycles, in a saturated Guanidinium HCl solution containing 1% CHAPS. FIG. 22A shows an agarose gel that demonstrates the presence of extracted genomic DNA in the crude lysate, purified using a QIAGEN® QIAamp DNA Tissue kit (the proteinase K digestion step was omitted).

Rat brain proteins were also extracted using PCT (five one-minute cycles at −25° C. in phosphate-buffered saline (PBS)), a no-pressure control, and by mortar and pestle in liquid nitrogen followed by Omni homogenization (FIG. 22B). The rat proteins were examined using Western blot analysis (FIG. 22C). In the Western Blot assay, the first antibody was the universal monoclonal anti-nitric oxide synthase, mouse; and the second antibody was anti-Mouse IgM (m-Chain specific) alkaline phosphatase conjugate (both antibodies were obtained from Sigma, St. Louis, Mo.). Brain tissue was homogenized in PBS.

This example demonstrates that PCT can extract DNA and proteins from an animal brain tissue. The PCT process was compatible with PCR amplification for DNA analysis. The antigenic reactivity of the nitric oxide synthase was also preserved. The ELISA assay (data not shown), also demonstrated that rat brain can be processed by PCT to yield other natural proteins.

### Other Embodiments

A number of embodiments of the invention have been described. Nevertheless, it will be understood that various modifications can be made without departing from the spirit and scope of the invention. Accordingly, other embodiments are within the scope of the following claims.

What is claimed is:

1. A sample processing device for use in a pressure-modulation apparatus, said device comprising:
multiple chambers;
at least one barrier positioned between two chambers, wherein said barrier is either porous or penetrable, or can be rendered porous or penetrable by exposure to a physical, chemical, or mechanical stimulus; and
at least one pressurizing member in a position to subject the sample to high pressure and to force the sample through at least one barrier.

2. The device of claim 1, wherein at least one of the chambers is part of a molded device.

3. The device of claim 1, comprising:
two or more chambers;
multiple barriers, at least one of which is porous or can be rendered porous or penetrable by exposure to a physical, chemical, or mechanical stimulus; and
two or more pressurizing members, which are activated independently, at differing hydrostatic pressures, having at least one member in a position to subject the sample to high pressure and to force the sample through at least one barrier.

4. The device of claim 3, wherein at least two of the chambers are linked together by threaded mechanical interlocks or a threading mechanism.

5. The device of claim 1, wherein at least one of the chambers comprises plastic, ceramic, metal or glass.

6. The device of claim 1, wherein at least one of the chambers has a volume up to 100 μl.

7. The device of claim 1, wherein at least one of the chambers has a volume up to 100 ml.

8. The device of claim 1, wherein at least one of the chambers has a volume up to 500 ml.

9. The device of claim 1, wherein the surface of one or more chambers is inert to biomolecules.

10. The device of claim 1, wherein the surface of one or more chambers is derivatized with biomolecules.

11. The device of claim 10, wherein the surface of one or more chambers is derivatized with molecules that interact with biomolecules.

12. The device of claim 11, wherein the molecules comprise small organic molecules.

13. The device of claim 1, wherein at least one barrier further comprises a filter.

14. The device of claim 13, wherein the filter comprises a material selected from the group consisiting of, polyvinyl chloride, polyether sulfone, nylon, nitrocellulose, cellulose esters, cellulose acetate, cellulose nitrate, polytetrafluoroethylene (PFTA), vinyl, polypropylene, and polycarbonate.

15. The device of claim 1, wherein said barrier is in a position between a first chamber and a second chamber, and openings in the barrier communicate between the first chamber and the second chamber.

16. The device of claim 1, wherein at least one barrier is pierceable.

17. The device of claim 1, wherein at least one barrier is capable of being dissolved by organic solvent.

18. The device of claim 1, wherein at least one barrier can be removed mechanically.

19. The device of claim 1, wherein at least one barrier can be removed through a change in temperature.

20. The device of claim 1, wherein at least one barrier comprises a valve.

21. The device of claim 1, wherein at least one barrier can be removed by any one or a combination of the following mechanisms: piercing, salvation, melting, breaking, and mechanical removal.

22. The device of claim 1, wherein said barrier comprises a polymer, metal, or ceramic.

23. The device of claim 1, wherein said barrier comprises a composite or layers of solid materials.

24. The device of claim 1, wherein said barrier has plurality of openings.

25. The device of claim 24, wherein said openings are generally round.

26. The device of claim 24, wherein the diameter of the openings is between about 1 μm and about 100 μm.

27. The device of claim 24, wherein the diameter of the openings are between 0.1 mm and 1 mm.

28. The device of claim 24, wherein the diameter of the openings are between 1 mm and 1 cm.

29. The device of claim 24, wherein the diameter of the openings are between 1 cm and 3 cm.

30. The device of claim 1, wherein the barrier contains a plurality of solid, pointed protrusions.

31. The device of claim 30, wherein said solid comprises a polymer, metal, or ceramic.

32. The device of claim 30, wherein said pointed protrusions are in the shape of a pyramid or cone.

33. The device of claim 30, wherein said protrusion extends 0.01 cm to 0.1 cm above the base of said screen.

34. The device of claim 30, wherein said protrusion extends 0.1 cm to 1 cm above the base of said screen.

35. The device of claim 30, wherein said protrusion extends, 1 cm to 3 cm above the base of said screen.

36. The device of claim 1, wherein said pressurizing member comprises at least one ram mounted to move within a chamber.

37. The device of claim 1, wherein the pressurizing member comprises a polymer, metallic, or ceramic material.

38. The device of claim 1, wherein the pressurizing member is circular in cross-section.

39. The device of claim 1, wherein the chambers comprise a wall and the pressurizing member comprises one or more annular seals in contact with the wall as the pressurizing member moves.

40. The device of claim 39, wherein said seal is polymeric, metallic, or ceramic.

41. The device of claim 1, wherein at least one pressurizing member comprises a cylinder having a sealing ring around its circumference, and the chambers are generally cylindrical.

42. The device of claim 1, wherein the device comprises at least two pressurizing members, one of the pressurizing members being positioned on a first side of a barrier and the other of the rams being positioned on a second side of the barrier, opposite to the first side of the barrier.

43. The device of claim 1, wherein more than one chamber is mounted with a pressurizing member.

44. The device of claim 1, further comprising a container having an orifice, said chambers being positioned within the orifice.

45. The device of claim 1, wherein one or more of the chambers is filled with a fluid.

46. The device of claim 1, wherein one or more of the chambers further comprises a temperature-controlling device.

47. The device of claim 1, wherein one or more of the chambers further comprises a temperature-cycling device.

48. The device of claim 1, wherein one or more of the chambers further comprises a pressure-controlling device.

49. The device of claim 1, wherein one or more of the chambers further comprises a pressure-cycling device.

50. The device of claim 1, further comprising an detection module built into one or more of the chambers.

51. A sample processing device for use in a pressure-modulation apparatus, said device comprising:
multiple chambers, wherein the chambers are positioned in a vertical configuration;
at least one temporary barrier positioned between pairs of chambers; and
one pressurizing member positioned to force a sample through at least one of the barriers.

52. The device of claim 51, further comprising a porous barrier positioned between the first and second chamber.

53. A method of processing a biological sample, comprising:
providing a device according to claim 1;
adding sample to a first chamber; and
subjecting the device to elevated pressure to cause the pressurizing member to force a sample through a barrier between said first chamber and second chamber, and into said second chamber.

54. The method of claim 53, wherein the biological sample is forced through multiple barriers by the pressurizing member.

55. The method of claim 53, wherein the biological sample is selected from the group consisting of; a solid material, a semi-solid material, a gas and a liquid.

56. The method of claim 53, wherein the biological sample is selected from the group consisting of; an insect or small animal, a fungus, a plant or animal tissue, a food or agricultural product, a forensic sample, and a human tissue.

57. The method of claim 53, wherein the biological sample comprises human blood, serum, or urine.

58. The method of claim 53, wherein the biological sample is frozen.

59. The method of claim 53, wherein the size of the biological sample is between 10 mg and 100 mg.

60. The method of claim 53, wherein the size of the biological sample is between 0.1 mg and 1.0 mg.

61. The method of claim 53, wherein the size of the biological sample is between 10 mg and 1 g.

62. The method of claim 53, wherein the size of the biological sample is between 1 g and 20 g.

63. The method of claim 53, wherein the size of the biological sample is between 20 g and 500 g.

64. The method of claim 53, wherein the elevated pressure is above 500 psi.

65. The method of claim 53, wherein the elevated pressure is above 5,000 psi.

66. The method of claim 53, wherein the elevated pressure is above 10,000 psi.

67. The method of claim 53, wherein the elevated pressure is above 50,000 psi.

68. The method of claim 53, wherein said elevated pressure is applied to the sample below the sample's atmospheric pressure freezing temperature.

69. The method of claim 53, wherein said elevated pressure is applied at a temperature in the range of 4° C. to ambient temperature.

70. The method of claim 53, wherein said elevated pressure is applied at ambient temperature.

71. The method of claim 53, wherein said elevated pressure is applied at a temperature in the range of ambient to 90° C.

72. The method of claim 53, wherein said elevated pressure is repeatedly cycled.

73. The method of claim 72, wherein said elevated pressure is cycled at a frequency in the range of milliseconds.

74. The method of claim 53, wherein said elevated pressure is cycled at a frequency in the range of seconds.

75. The method of claim 53, wherein said elevated pressure is cycled at a frequency in the range of minutes.

76. The method of claim 53, further comprising analyzing the sample after, or as part of, said sample preparation.

77. The method of claim 53, further comprising extracting a specific substance or substances from the biological sample.

78. The method of claim 53, wherein DNA, RNA, or at lease one cellular protein in said sample is isolated after or as part of said sample preparation.

79. The method of claim 53, wherein a pharmaceutical composition is isolated from the biological sample after or as part of said sample preparation.

80. The method of claim 53, further comprising processing a specific substance or substances present in from the biological sample.

81. The method of claim 80, wherein the processing step is selected from the group consisting of; amplification of a specific substance, specific binding to a ligand, specific elution from a ligand, carrying out a chemical reaction, carrying out one or more enzymatic reactions, carrying out one or more nucleic acid sequencing reactions, solubilization of recombinant proteins from inclusion bodies, and carrying out one or more catalytic reactions.

82. The method of claim 81, wherein the amplification is carried out using polymerase chain reaction.

83. The method of claim 81, wherein the chemical reaction comprises nucleic acid hybridization.

84. The method of claim 81, wherein the chemical reaction comprises interacting an antigen and antibody.

85. The method of claim 80, wherein the processing comprises carrying out stepwise reactions, wherein a different step takes place in each chamber of the device.

86. The method of claim 53, wherein multiple devices according to claim 1 are interconnected in said pressure-modulation apparatus.

87. The method of claim 86, wherein 8 to 12 devices according to claim 1 are interconnected together to form a strip.

88. The method of claim 86, wherein multiple devices according to claim 1 are interconnected to form a two-dimensional matrix of devices.

89. The device of claim 1, wherein one or more chambers containing a reagent annularly surround a chamber containing a sample, and wherein said reagent is introduced to the sample through a valve activated by pressure.

90. The device of claim 1, wherein at least one of the chambers is equipped with electrodes enabling electric current to be passed through a chamber during processing of the sample.

91. The device of claim 1, further comprising a cap at an end of the device.

92. The device of claim 91, wherein the cap is linked to a chamber at an end of the device by threaded mechanical interlocks or a threading mechanism.

93. The device of claim 91, wherein the cap can be removed by any one or a combination of the following mechanisms: piercing, solvation, melting, breaking, and mechanical removal.

94. The device of claim 92, wherein the chamber at the end of the device comprises a wall and the cap further comprises one or more annular seals in contact with the wall when the cap is linked to the chamber.

95. The method of claim 53, further comprising maintaining the sample in the device prior to subjecting the device to elevated pressure, for a length of time required to store the sample at a temperature appropriate to store the sample.

96. The method of claim 53, further comprising maintaining the sample in the device subsequent to subjecting the device to elevated pressure for a length of time required to store the sample at a temperature appropriate to store the sample.

97. The device of claim 50, wherein the detection module is selected from the group consisting of, a luminometer, a fluorometer, a photometer, a spectrophotometer, an ionization detector, a flow counter, a scintillation counter, and a camera.

98. The device of claim 1, wherein at least one of the chambers has a volume up to 500 μl.

99. The method of claim 53, wherein the size of the biological sample is between 1.0 mg and 10 mg.
